(19) United States
(12) Patent Application Publication
(10) Pub. No.: US 2010/0210819 A1
(43) Pub. Date: Aug. 19, 2010

(54) Biosensors to measure InsP3 concentration in living cells

(76) Inventors: Gregory A. Mignery, Naperville, IL (US); Timothy Paul Remus, Chicago, IL (US)

Correspondence Address: DONNA BOBROWICZ;LOYOLA UNIVERSITY CHICAGO,, STRITCH SCHOOL OF MEDICINE, BLDG 120, ROOM 400, 2160 S. FIRST AVE., MAYWOOD, IL, 60153, US

(21) Appl. No.: 11/807,528

(22) Filed: May 25, 2007

Related U.S. Application Data
(60) Provisional application No. 60/808,268, filed on May 25, 2006.

(51) Int. Cl. C07K 14/705; C12N 15/12; C12N 15/85

(52) U.S. Cl. 530/350; 536/23.5; 435/320.1

(57) ABSTRACT

Phosphoinositides participate in many signaling cascades via phospholipase C stimulation, which hydrolyzes phosphatidylinositol bisphosphate, producing second messengers diacylglycerol and inositol 1,4,5-trisphosphate (InsP₃). Destructive chemical approaches required to measure [InsP₃] limit spatio-temporal understanding of subcellular InsP₃ signaling. Disclosed are new biosensors and test kits which allow studying InsP₃ dynamics at high temporal and spatial resolution, thereby understanding InsP₃ signaling in intact cells.

### CROSS-REFERENCE TO RELATED APPLICATIONS

This application claims the benefit of the filing date of U.S. Provisional Application No. 60/808,268, filed on May 25, 2006, which is herein incorporated by reference.

### RIGHTS OF THE US GOVERNMENT TO THIS INVENTION

Supported by National Institutes of Health grants MH53367, HL30077 and HL64724, HL62231. The United States government may have certain rights in this invention.

### FIELD OF THE INVENTION

New fluorescence based biosensors are disclosed which allow studying InsP₃ concentration dynamics in intact cells at high temporal and spatial resolution. These inventive biosensors can also be used in vitro as a rapid fluorometric based InsP₃ quantification assay.

### BACKGROUND OF THE INVENTION

Cell surface membrane receptor-activation of phospholipase C (“PLC”) results in hydrolysis of phosphatidylinositol (4,5) bisphosphate (“PIP₂”) and the production of the second messengers diacylglycerol and inositol 1,4,5-trisphosphate (“InsP₃”). This signaling step is recognized as a crucial branch point in signal transduction where membrane delineated diacylglycerol modulates protein kinase C (“PKC”), while InsP₃ can diffuse into the cytoplasm and mediate calcium (“Ca”) release from intracellular stores via the InsP₃ receptor (“InsP₃R”).

The second messenger InsP₃ occupies a central position in the initiation and propagation of intracellular Ca release through InsP₃Rs that regulate a myriad of cellular events. Until very recently the real time analysis of InsP₃ liberation, concentration dynamics, and spatial distribution in a living cell have not been possible. Measurements have relied upon destructive methodologies, either whole cell extracts for mass analysis by competition binding, gas chromatography/mass spectroscopy, and ion exchange chromatography, or use metabolic measurements of radiolabeled InsP₃ precursors and degradation products. (Challiss, R. A., Chilvers, E. R., Willcocks, A. L., and Nahorski, S. R., Biochemical Journal 265, 421-427 (1990); Dean, N. M. and Beaven, M. A., Analytical Biochemistry 183, 199-209 (1989); Nahorski, S. R., Young, K. W., John Challiss, R. A., and Nash, M. S., Trends in Neurosciences 26, 444-452 (2003); Woodcock, E. A., Molecular & Cellular Biochemistry 172, 121-127 (1997)). These methods unfortunately are of limited utility in deciphering the spatio-temporal organization of this second messenger system at the cellular and subcellular level. Recently, novel fluorescent probes have been developed to study intracellular InsP₃ dynamics. For example, a type-3 InsP₃R derived biosensor called LIBRA was employed to measure InsP₃ concentrations in SH-SY5Y cultured cells, which represents an important first step in the generation of physiologically relevant reagents to evaluate InsP₃ in living cells (Tanimura, A., Nezu, A., Morita, T., Turner, R. J., and Tojyo, Y. Journal of Biological Chemistry 279, 38095-38098 (2004)). Additionally, a plekstrin homology domain from PLCδ1-GFP fusion (“PHD-GFP”) was developed (Sugimoto, K., Nishida, M., Otsuka, M., Makino, K., Ohkubo, K., Mori, Y., and Morii, T. Chemistry & Biology 11, 475-485 (2004)) to evaluate plasma membrane PIP₂ concentration dynamics using a cytoplasmic translocation assay. It was found that this construct bound InsP₃ at high affinity and could be used to estimate [InsP₃]ᵢ. (Use of brackets “[ ]” around words in this document denote concentration). With a similar approach agonist-induced oscillatory changes of [InsP₃] could be measured in Chinese Hamster Ovary cells (Bartlett, P. J., Young, K. W., Nahorski, S. R., and Challiss, R. A. Journal of Biological Chemistry 280, 21837-2184613 (2005)). Although these approaches represent remarkable progress for the study of the subcellular dynamics of InsP₃ signaling, they either lack specificity and sensitivity, were only assessed in generic cultured cells, or are restricted to the measurement of [InsP₃] in specific subcellular, membrane bound, domains of the cell. Since there is increasing appreciation of local spatial compartmentalization and microdomains of intracellular signaling, the ability to measure local [InsP₃] in living cells would be highly beneficial in unraveling InsP₃-dependent signaling.

Green fluorescent protein (“GFP”) and its variants with different spectral characteristics have been used in the development of novel biosensors that can be expressed in living cells. Moreover, fusion proteins, such as those that include both a cyan and yellow fluorescent protein (“CFP” and “YFP”), are capable of exhibiting fluorescence resonance energy transfer (“FRET”) from CFP to YFP. By inserting peptide linkers that bind to biological molecules of interest, biosensors whose FRET properties change upon binding the molecule of interest can be developed. (Miyawaki, A., Llopis, J., Heim, R., Mccaffery, J. M., Adams, J. A., Ikura, M., and Tsien, R. Y. Nature 388, 882-887 (1997); Tanimura, A., Nezu, A., Morita, T., Turner, R. J., and Tojyo, Y. Journal of Biological Chemistry 279, 38095-38098 (2006); Zhang, J., Ma, Y., Taylor, S. S., and Tsien, R. Y. Proceedings of the National Academy of Sciences of the United States of America 98, 14997-15002 (2001)).

Recently, progress has been made to use fluorescent probes to measure [InsP₃] dynamically in living cells (Tanimura, A., Nezu, A., Morita, T., Turner, R. J., and Tojyo, Y. Journal of Biological Chemistry 279, 38095-38098 (2006); Bartlett, P. J., Young, K. W., Nahorski, S. R., and Challiss, R. A. Journal of Biological Chemistry 280, 21837-21846 (2005); Sugimoto, K., Nishida, M., Otsuka, M., Makino, K., Ohkubo, K., Mori, Y., and Morii, T. Chemistry & Biology 11, 475-485 (2004)). A key approach is to use the InsP₃ binding domain of the InsP₃ receptor, because this is one of the crucial functional targets in the cell. However, more sensitive sensors are needed to measure, in a non-destructive manner, local InsP₃ concentration in living cells. Additionally, an assay kit measuring enhanced sensitivity and dynamic ranges is desirable to the research scientist.

### SUMMARY OF THE INVENTION

The present invention provides for new InsP₃ binding FRET-based sensors using the ligand binding domains of the type-1 and type-3 InsP₃R isoforms (“FIRE-1” and “FIRE-3”) (SEQ ID NO 1 and SEQ ID NO 2). These sensors utilize the InsP₃R type-1 and type-3 ligand binding domains expressed as chimeras terminally linked to CFP and YFP fluorescent proteins. It is believed to be within the scope of those knowledgeable in the art to replace these fluorescent proteins with variant pairs having appropriate FRET properties and these variant pairs are also considered to be encompassed by the present invention. The invention comprises the nucleotide sequence encoding a genetically engineered biosensor protein comprising a binding ligand of InsP₃ and one fluorescent molecule on the amino terminus and a different fluorescent molecule on the carboxyl terminus, having a nucleotide sequence of SEQ ID NO 1 or of SEQ ID NO 2. The invention also comprises a genetically engineered protein comprising an amino acid sequence of a binding ligand having two termini, each terminus having a different fluorescent molecule whereby upon coming into proximity with each other exhibit fluorescence resonance energy transfer, wherein said amino acid sequence is substantially identical to the sequence selected from the group consisting of SEQ ID NO 6 and SEQ ID NO 7. These biosensors have been coined FIRE (fluorescent InsP₃ Responsive

Element). They are expressed as soluble proteins and are uniformly distributed throughout the cytoplasm of all cells tested. The present inventors have characterized FIRE-1 and FIRE-3 in solution and in cultured mammalian cells (COS-1) and acutely isolated primary cells, cardiac myocytes, exposed to agonists expected to raise InsP₃ production.

In addition to sensing intracellular changes of InsP₃, the FIRE expression products are functional in vitro and respond to incremental additions of InsP₃. This property enables a dual use for these recombinant proteins in that FIRE expression products can be used in a rapid in vitro fluorometric assay to measure samples containing unknown concentrations of InsP₃. An in vitro kit comprising the FIRE expression products that are affinity purified using established methodologies, resulting in enhanced sensitivities and dynamic ranges.

### DESCRIPTION OF THE DRAWINGS

FIG. 1. A) FIRE schematic indicating junctional linker sequences between the fluorescent proteins and the InsP₃R ligand binding region. B) Western immunoblotting of InsP₃-sensor (FIRE) expression products from COS and adenoviral infected adult cardiac myocytes.

FIG. 2. In vitro calibration and inositol phosphate specificity of FIRE biosensors. A) [Ins(1,4,5)P₃]-dependence of FRET expressed as changes in F₅₃₀/F₄₈₀ for FIRE-1 (top) and FIRE-3 (bottom). B) FIRE-1 activation by adenophostin A, heparin and other inositol phosphates. The apparent affinity of each compound for FIRE-1 is indicated.

FIG. 3. A) Single cell [Ca]; measurement using the fluorescent Ca indicator fluo-4/AM. Changes in [Ca]; are expressed as F/F₀ and were elicited by extracellular ATP (10 μM) stimulation. B) Changes in [InsP₃] elicited by stimulation with extracellular ATP (10 μM). Changes in [InsP₃] are expressed as % changes of F₅₃₀/F₄₈₈ relative to the level encountered prior to the application of ATP. Inset: Photomicrograph of FIRE-1 YFP fluorescence in a cultured COS-1 cell (excitation 457 nm, emission >530 nm). C) Average changes of [InsP₃] (% change of F₅₃₀/F₄₈₈) in COS-1 cells expressing FIRE-1 or FIRE-3 upon stimulation with ATP (10 μM) or acetylcholine (5 μM). Numbers in parentheses indicate number of individual cells tested under each experimental condition.

FIG. 4. Acceptor (YFP) photobleach in COS-1 expressing FIRE-1 and FIRE-3. A, left column, CFP and YFP basal fluorescence. The cell expressing FIRE-1 was subsequently exposed to laser light of 514 nm at maximal available intensity for 10-30 s to achieve acceptor (YFP) photobleaching. Right column, CFP and YFP fluorescence after photobleach. B, average increase of donor (CFP) fluorescence after photobleach, indicative of FRET between CFP and YFP in FIRE-1 and FIRE-3. For CFP, excitation (Ex) was 457 nm, and emission (Em) was 488 nm; For YFP, excitation was 514 nm, and emission was 530 nm.

FIG. 5. Increase of [InsP₃] in neonatal ventricular myocytes by stimulation withendothelin-1. ET-1 (100 nM) induced an transient increase of [InsP₃]. Changes in [InsP₃] are expressed as the percentages of change of F530/F488. The average data from four cells are shown. Inset, FIRE-1 YFP fluorescence in a cultured neonatal ventricular cell (excitation, 457 nm; emission, 530 nm).

FIG. 6. Acceptor (YFP) photobleach in adult cat ventricular myocytes expressing FIRE-1. A, FIRE-1 was adenovirally expressed (FIRE-1-AdV) in acutely isolated cat ventricular myocytes. The same photobleach protocol as in FIG. 4 was applied to intact adult myocytes. B, photobleach protocol applied to cells internally perfused with InsP₃ (10 μM) through a patch pipette. C, average percentage increases in donor (CFP, blue) and decreases in acceptor (YFP, yellow) fluorescence in intact (left) cells and in cells internally perfused with InsP₃ (right). The numbers in parentheses indicate the numbers of individual cells tested under each experimental condition. Ex, excitation; Em, emission.

FIG. 7. Agonist induced changes of [InsP₃] in adult ventricular myocytes. A) Adult ventricular myocytes were stimulated with endothelein-1 (ET-1, 100 nM; top), phenylephrine (Phe, 10 μM; middle) and angiotensin II (Ang II, 10 μM; bottom). B) Internal perfusion of ventricular myocytes with an internal solution containing a saturating concentration of InsP₃ (10 μM) and internal solution containing no InsP₃. C) Average % changes in [InsP₃] (F₅₃₀/F₄₈₈) elicited by agonist stimulation or internal perfusion with or without InsP₃. Numbers in parentheses indicate number of individual cells tested under each experimental condition.

FIG. 8. Spatial [InsP₃] gradients in adult ventricular myocytes. A) Top: Spatial distribution of FIRE-1 (excitation 457 nm; emission >530 nm) in an adult cat ventricular myocyte. The rectangles marked 1-4 indicate regions of interest (ROIs) from where the traces shown in panel B were recorded. Bottom: Representation of the spatial distribution of [InsP₃]. The middle panel shows a spatial InsP₃ diffusion profile along the longitudinal axis of the cell recorded from the ROI marked by the rectangle to the left of the bottom image. B) Time course of changes of [InsP₃] at different subcellular locations after rupturing the membrane and begin of internal InsP₃ perfusion. ROI 1 (black symbols) directly underneath the patch pipette; ROIs 3 (red) and 4 (green) are cytosolic at different distance from the perfusion pipette; ROI 2 (blue) represents the cell nucleus. Inset: time (t_{1/2}) required to reach half-maximal levels of [InsP₃] as a function of distance from the perfusion pipette (2 experiments). Open symbols: cytosol; closed symbols: nucleus.

FIG. 9. Spatial [InsP₃] gradients in an intact adult ventricular myocyte stimulated with endothelin-1. A) Photomicrograph of spatial distribution of FIRE-1 in an adult cat ventricular myocyte. Ovals mark nuclear (black) and cytosolic (white) ROI. B) Time course of increase of nuclear (closed symbols) and cytosolic (open symbols) [InsP₃] (expressed as % change of F₅₃₀/F₄₈₈) elicited be exposure to ET-1 (100 nM). The gray lines represent exponential fits to the data. C) Average magnitude (left) and time course (t_{1/2}; right) of increase of [InsP₃] (expressed as % change of F₅₃₀/F₄₈₈). Numbers in parentheses indicate number of individual cells tested under each experimental condition.

FIG. 10. The FIRE-1 plasmid.

FIG. 11. The FIRE-3 plasmid.

FIG. 12. Nucleotide assignments of FIRE plasmid inter-domain boundaries as further seen in FIGS. 13 and 14.

FIG. 13. Nucleotide sequence of FIRE-1. SEQ ID NO 6. Coding region in SEQ ID NO 1.

FIG. 14. Nucleotide sequence of FIRE-2. SEQ ID NO 7. Coding region in SEQ ID NO 2.

### DETAILED DESCRIPTION OF THE INVENTION

The construction and characterization of prototypic fluorescent biosensors, FIRE-1 and FIRE-3, that allow quantitative measurement of cellular InsP₃ levels in a living cell with temporal and spatial resolution are described. FIRE-1 and FIRE-3 utilize the InsP₃R type-1 and type-3 ligand binding domains expressed as chimeras terminally linked to CFP and YFP fluorescent proteins. Other variant pairs of fluorescent proteins, such as GFP and mCherry or citrine and CFP may also be useful in the present invention, as long as they exhibit the appropriate FRET properties. The biosensors are expressed as soluble proteins and are uniformly distributed throughout the cytoplasm of all cells tested. In vitro fluorimetric characterization of FIRE-1 and FIRE-3 show that they respond by exhibiting increased FRET upon incremental additions of InsP₃ with an enhanced dynamic range and with a superior sensitivity (˜42× higher apparent affinity) than the LIBRA sensor described above. The LIBRA sensor exhibits a decrease in FRET in response to InsP₃ binding whereas the inventive sensors demonstrate an increase in FRET. In LIBRA, it is hypothesized that this is a consequence of the plasma membrane targeting sequence present in LIBRA, and that membrane insertion results in a conformation that positions the two fluorophors in proximity which is reduced upon ligand binding. In contrast, both FIRE-1 and FIRE-3 have very similar concentration-dependent FRET responses even though the reported apparent affinities for InsP₃ for InsP₃R1 and InsP₃R3 differ considerably (Newton, C. L., Mignery, G. A., and Sudhof, T. C. J Biol Chem 269, 28613-28619 (1994)). However, those affinity measurements were made in the context of either larger fragments or whole receptor protein and not chimeric assemblies terminally linked to fluorescent proteins.

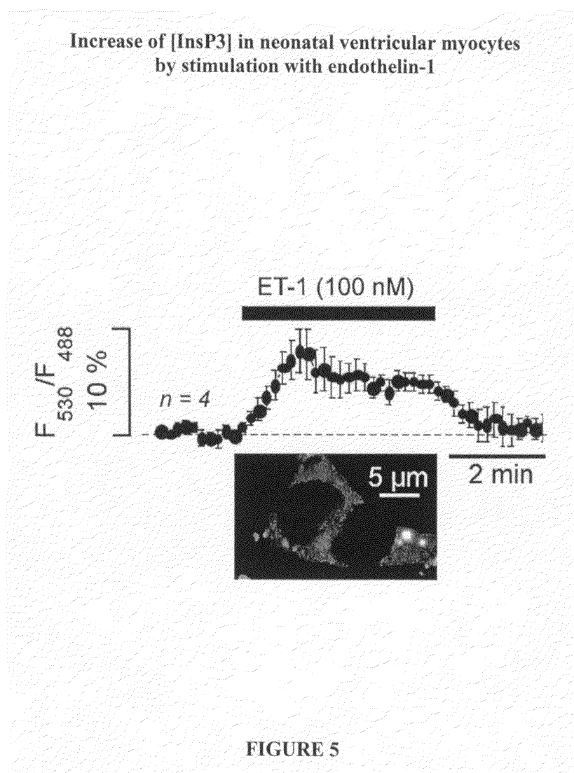

Analysis of the response of FIRE-1 to other inositol-phosphates reveals that they react very similarly to the intact InsP₃R. The two primary products of cellular InsP₃ metabolism, Ins(1,3,4,5)P₄ and Ins(1,4)P₂, as well as the other inositol-phosphates examined (Ins(2,4,5)P₃, Ins(4,5)P₂) interacted with FIRE-1 consistent with previous competition binding and Ca release studies (Wilcox, R. A., Primrose, W. U., Nahorski, S. R., and Challiss, R. A. Trends in Pharmacological Sciences 19, 467-475 (1998); Sudhof, T. C., Newton, C. L., Archer, B. T., 3rd, Ushkaryov, Y. A., and Mignery, G. A. Embo J 10, 3199-3206 (1991); Wilcox, R. A., Challiss, R. A., Liu, C., Potter, B. V., and Nahorski, S. R. (1993) Molecular Pharmacology 44, 810-817; Lu, P. J., Gou, D. M., Shieh, W. R., and Chen, C. S. (1994) Biochemistry 33, 11586-11597). As expected, Ins(1,4)P₂, which has no activity in Ca-signaling (Berridge, M. J., and Irvine, R. F. Nature 341, 197-205 (1989)) did not induce FRET in cytosolic extracts expressing FIRE-1. The other major metabolite of InsP₃ via the 3-kinase, Ins(1,3,4,5)P₄, induced an increase in FRET with FIRE-1 yet at a significantly lower apparent affinity (563 nM) compared to InsP₃ (˜31 nM). The likelihood that [InsP₃]ᵢ are over-estimated as a consequence of Ins(1,3,4,5)P₄ accumulation, for example in the agonist-induced FRET whole cell experiments, is low since the predominant pathway for InsP₃ degradation is through the 5-phosphatase. Although the 3-kinase has a relatively high affinity for InsP₃ (sub to low micromolar range), its V. is significantly less than the 5-phosphatase (Shears, S. B. Advances in Second Messenger & Phosphoprotein Research 26, 63-92 (1992)). Furthermore, the rapid decline of the FRET signal after removal of the agonist endothelin-1 (“ET-1”), as seen in FIG. 5, strongly suggests that FIRE-1 reports changes in [InsP₃] rather than accumulating InsP₃ degradation products. Accordingly, the specificity of the FIRE sensor is high and effectively mimics the same specificity and selectivity for InsP₃ that the intracellular receptor InsP₃R exhibits. Thus, the FIRE-1 sensor can not only be used to evaluate cellular [InsP₃], but may be of significance as an indicator of InsP₃R activation independent of Ca release due to altered channel gating.

The effectiveness of FIRE in measuring the liberation of InsP₃ in culture cell lines as well as in acutely isolated cells has been demonstrated. In addition to the quantitative qualities of this indicator, our results show that these sensors are readily able to resolve the temporal nature of agonist induced InsP₃ generation. In the cellular context these sensors have a very similar dynamic range to that observed in vitro.

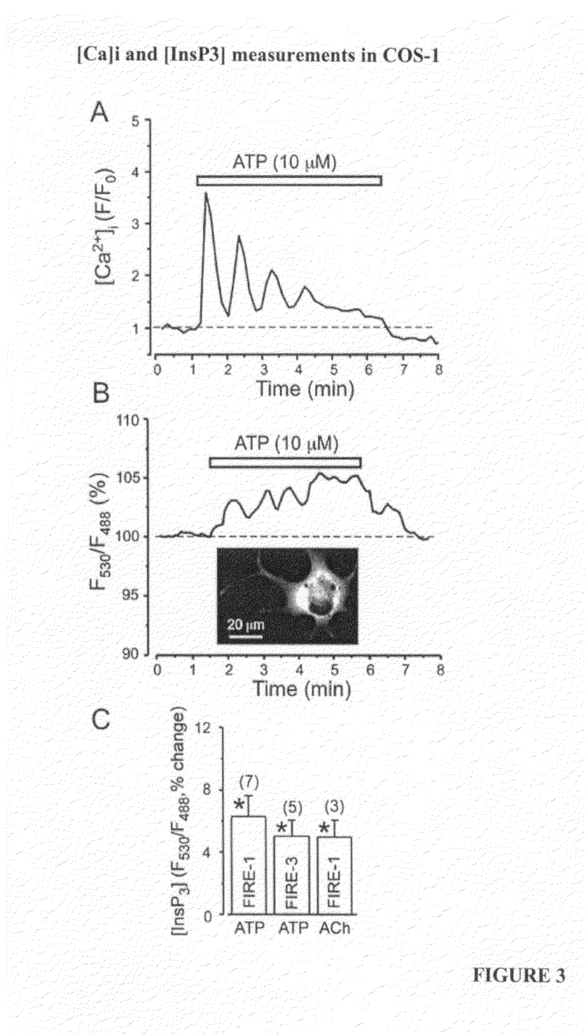

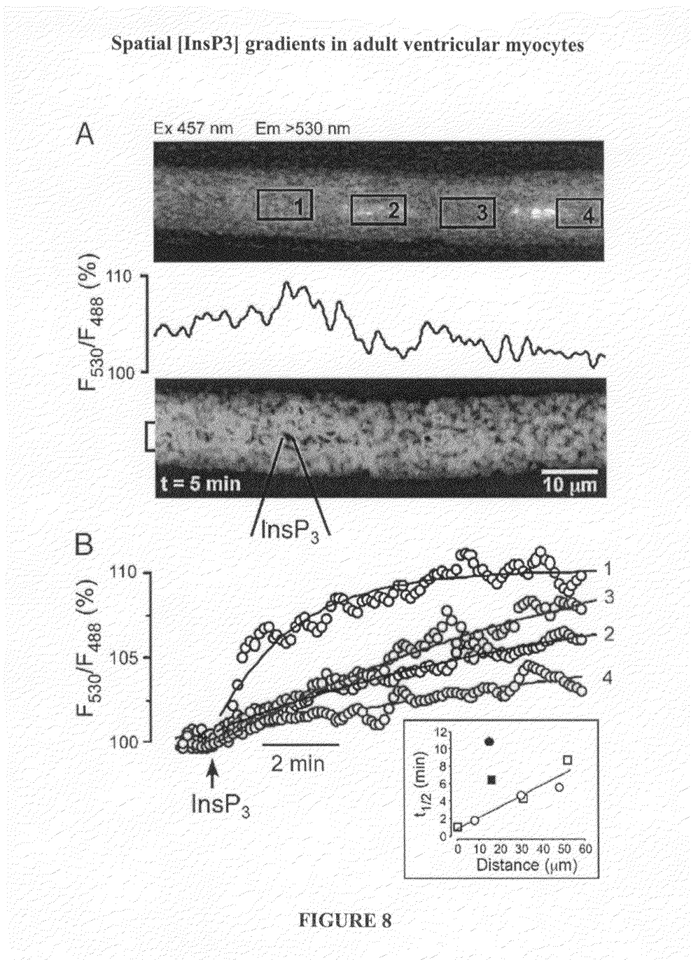

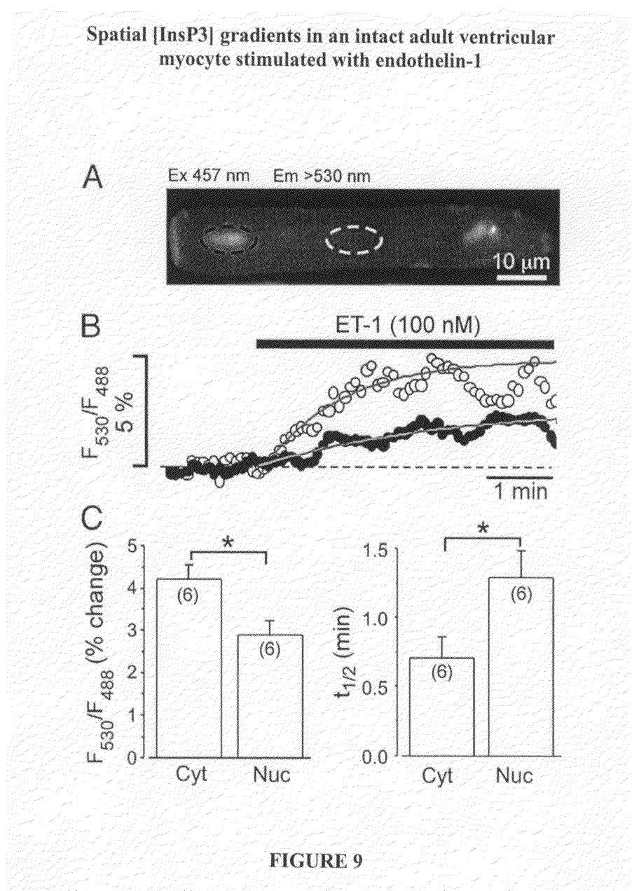

InsP₃ perfusion and agonist stimulation experiments in adult cardiac myocytes infected with FIRE-1 adenovirus (“AdV”) demonstrate that [InsP₃]ᵢ can be deduced, and that the spatial properties of the second messenger signal as a function of the ubiquitous distribution of FIRE in the cell can be measured. This is considered advantageous over the membrane associated LIBRA expression product or the plekstrin homology domain-GFP construct that relies on translocation of the indicator from the plasma membrane to the cytosol bound to InsP₃ which may impinge upon the second messengers diffusion. FIGS. 8 and 9 also reveal that InsP₃ diffuses into the nucleus of the myocytes with a delay and reaches lower levels than in the cytosol. This suggests that the spatio-temporal pattern of nuclear InsP₃ signaling differs from the cytosol. This is an important finding with regard to the specific role of InsP₃R type-2 localized to the nuclear envelope. For example, on the nuclear envelope InsP₃R2 associates with Ca/calmodulin-dependent protein kinase II (“CaMKII”) δ_{B} and this protein complex has been implicated in specific functions in cardiac myocytes (Wu, X., Bossuyt, J., Zhang, T., Mckinsey, T., Brown, J. H., Olsen, E. N., and Bers, D. M. Circulation 110 (supplement), 28 (2004)). In cardiac muscle, for example, FIRE sensors will be important tools to study the kinetics and localization of [InsP₃] during events that induce translocation of the transcription factor histone deacetylase (“HDAC”) as well to characterize spatio-temporal patterns of InsP₃ production involved in neurohumoral stimulation of cardiac myocytes during E-C coupling and NFAT translocation in hypertrophy and heart failure. In other cells where InsP₃ directs oscillatory Ca transients (e.g., FIG. 3) this sensor will allow determination of whether the Ca oscillations are driven by InsP₃ oscillations. The current FIRE constructs are capable of being subcellularly targeted to discreet localizations for measurement of InsP₃ generation in specific subcellular microdomains or as a complementary spatio-temporal indicator relative to other indicators, such as Ca indicators to characterize the interplay between Ca and InsP₃ signaling pathways.

To provide a way to better understand InsP₃ signaling in cardiac myocytes, FIRE-1 was incorporated in an adenovirus. This allowed successful tracking of agonist-induced subcellular [InsP₃] changes with high spatial and temporal resolution in ventricular myocytes.

### Experiments

Reagents: The D-myo-inositol phosphates were purchased from the following vendors: Ins(1,4,5)P₃ Alexis (San Diego, Calif.) Ins(2,4,5)P₃ from Calbiochem (LaJolla, Calif.), Ins(1,4)P₂, Ins(4,5)P₂ and Ins(1, 3, 4,5)P₄ A. G. Scientific (San Diego, Calif.). D-myo-Ins(1,4,5)P₃ and adenophostin A were obtained from Calbiochem (LaJolla, Calif.). Heparin was from Sigma Aldrich (St. Louis, Mo.).

Construction of FIRE plasmids: Type-1 and -3 InsP₃R biosensors were assembled using the individual ligand binding domain terminally fused with enhanced CFP (eCFP) and YFP (eYFP) at the amino and carboxyl termini, respectively. The construction of the FIRE plasmids corresponding to the two InsP₃R isoforms was as follows: The ligand binding regions of each receptor isoform (Hirata, M., Suematsu, E., Hashimoto, T., Hamachi, T., and Koga, T. Biochemical Journal 223, 229-236 1984) encompassing the amino-terminal 604 residues (589 for the type 1 InsP₃R SI⁻ isoform) were PCR amplified using the following oligonucleotide primer pairs: SEQ ID NO 3: GGAGATCTCGAGCTATGTCTGACAAAATGTC/SEQ ID NO 4: CGCGGATCCTTTCGGTTGTTGTGGAGCAG (Type-1); and SEQ ID NO 5: GGAGATCTCGAGCTATGAATGAAATGTCCAGC/SEQ ID NO 4: CGCGGATCC-TTTCGGTTGTTGTGGAGCAG (Type-3). Rat sequences for the three isoforms corresponding to GenBank accession numbers J05510, X61677 and L06096 were used as templates for the PCR reactions.

The PCR products from the individual InsP₃R isoforms were inserted into a pECYFP vector. This vector was constructed by linearizing pECFP-C1 (Clonetech, BD Biosciences) isolated from a methylation deficient E. coli strain (DM(-)) with Xba I and inserting the Xba I fragment of pEYFP (Clonetech, BD Biosciences) containing the eYFP coding sequence.

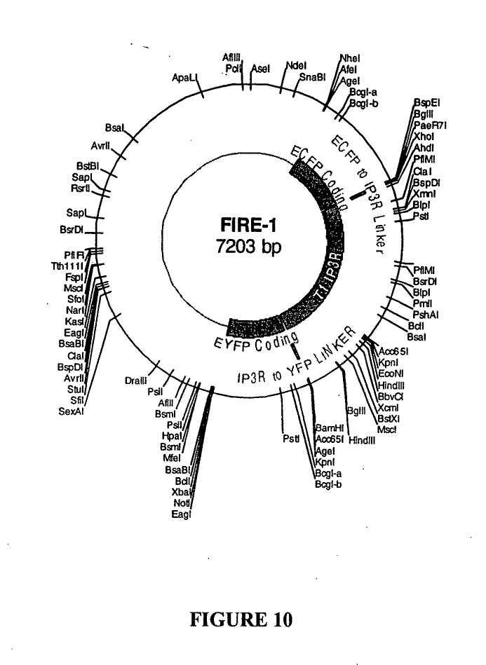

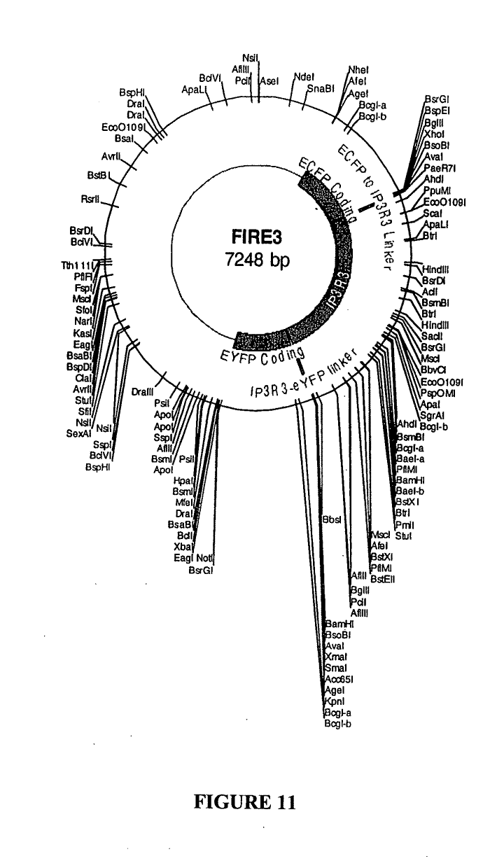

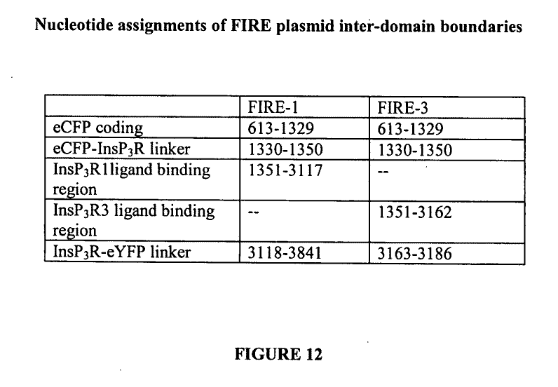

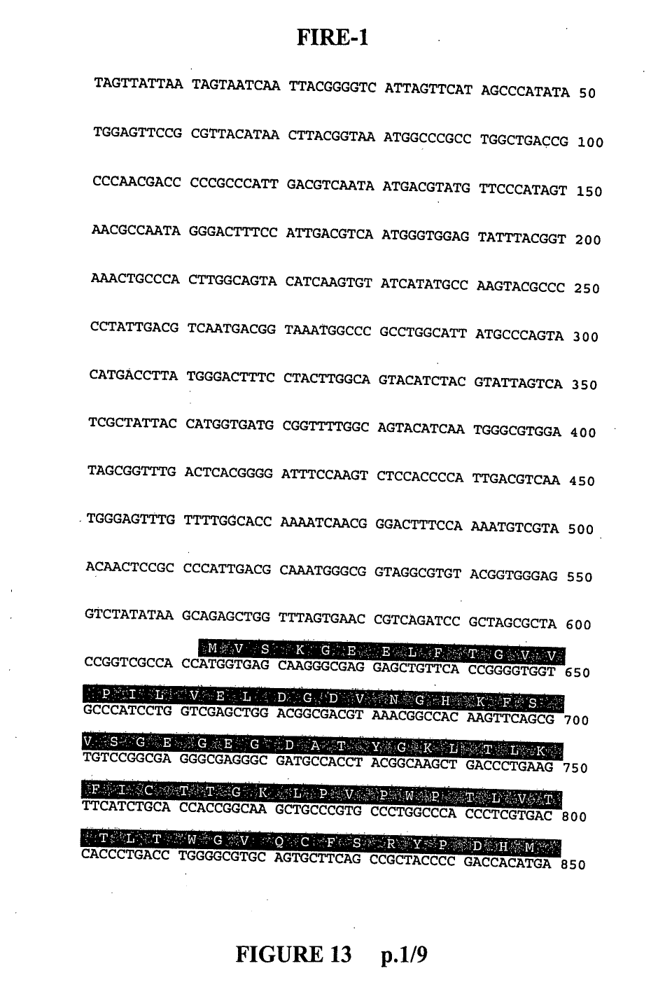

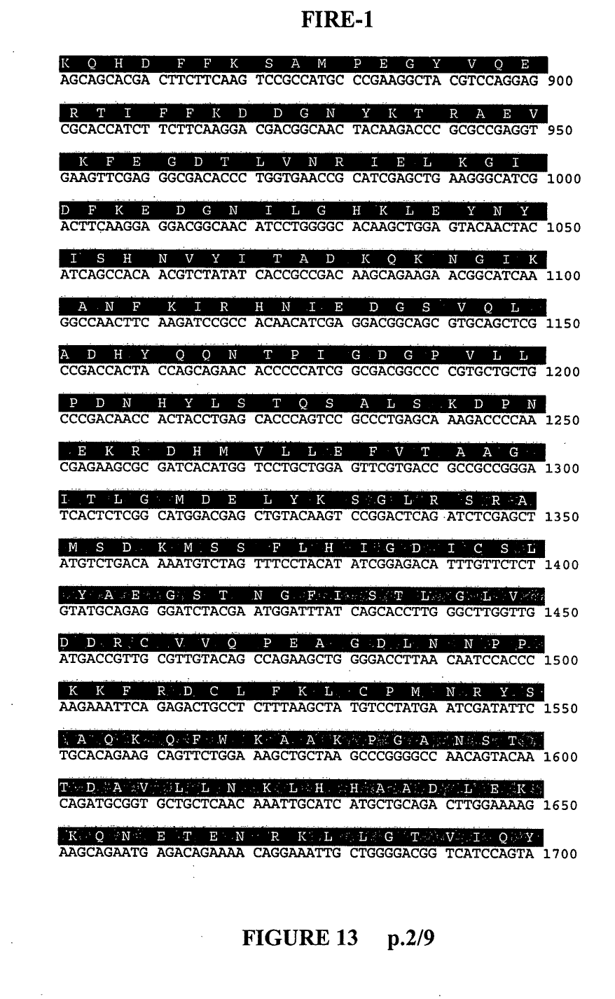

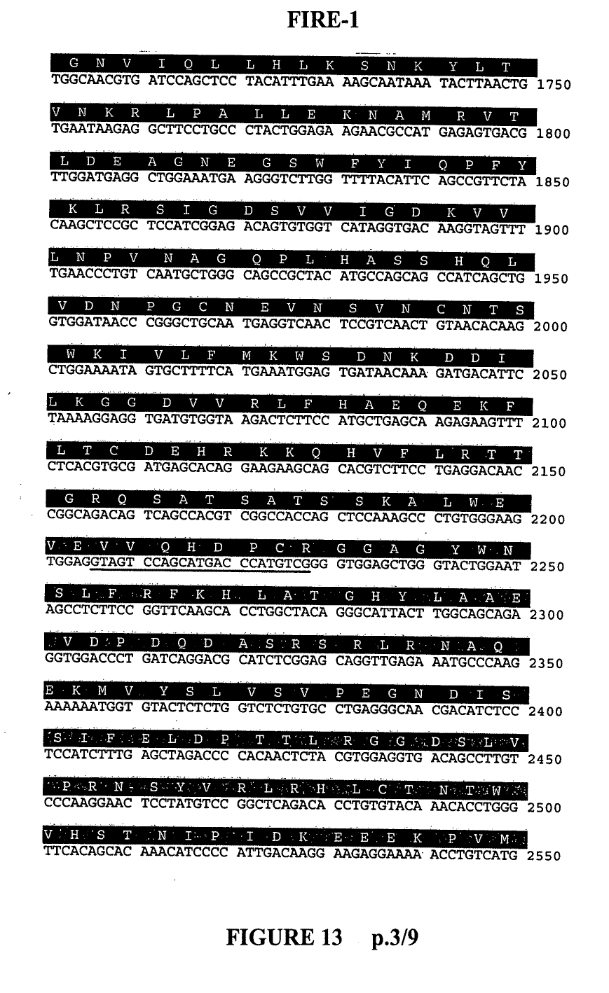

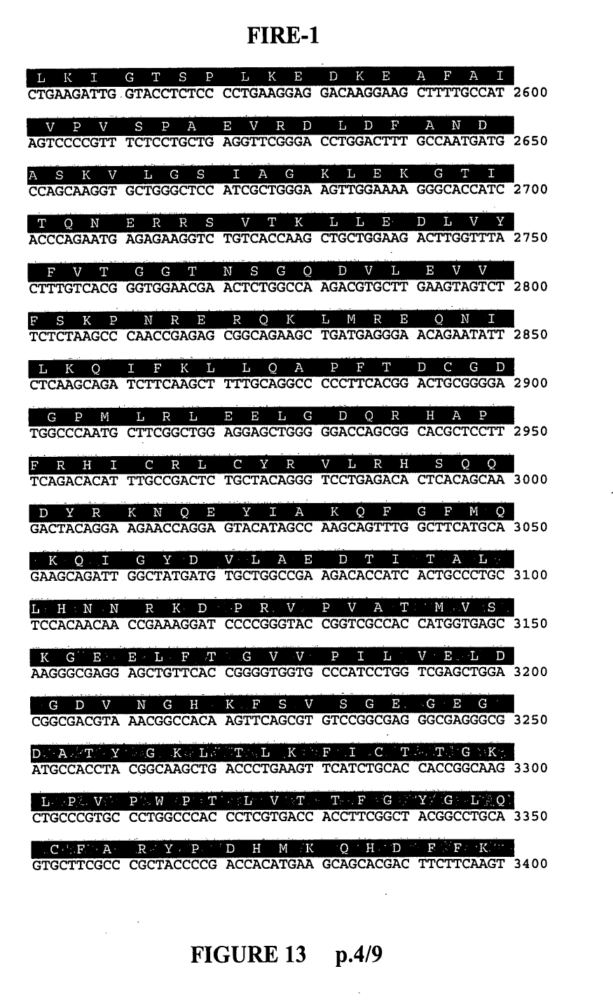

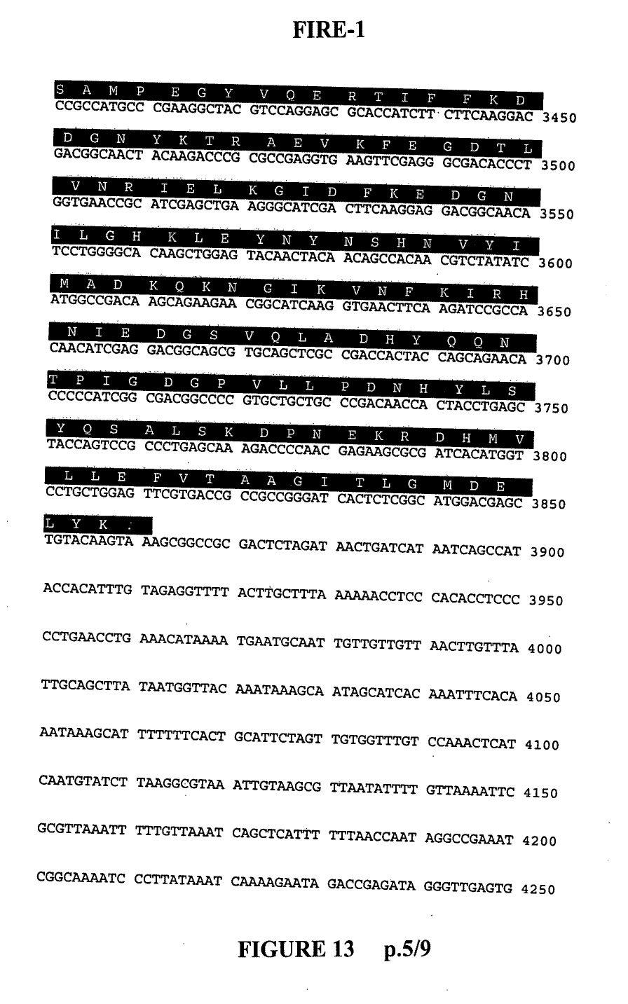

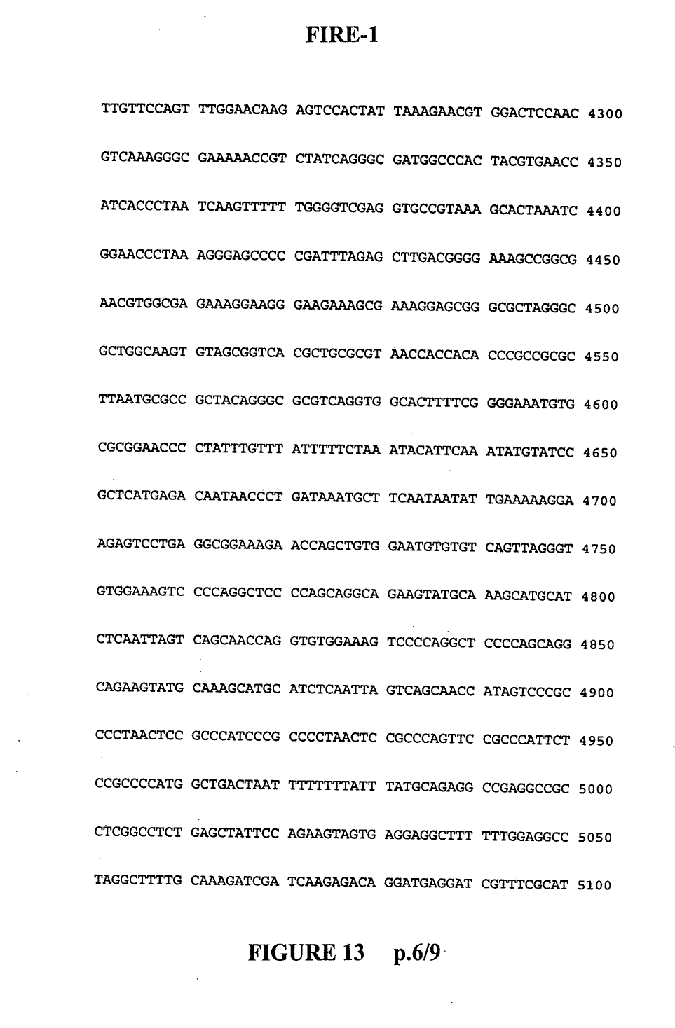

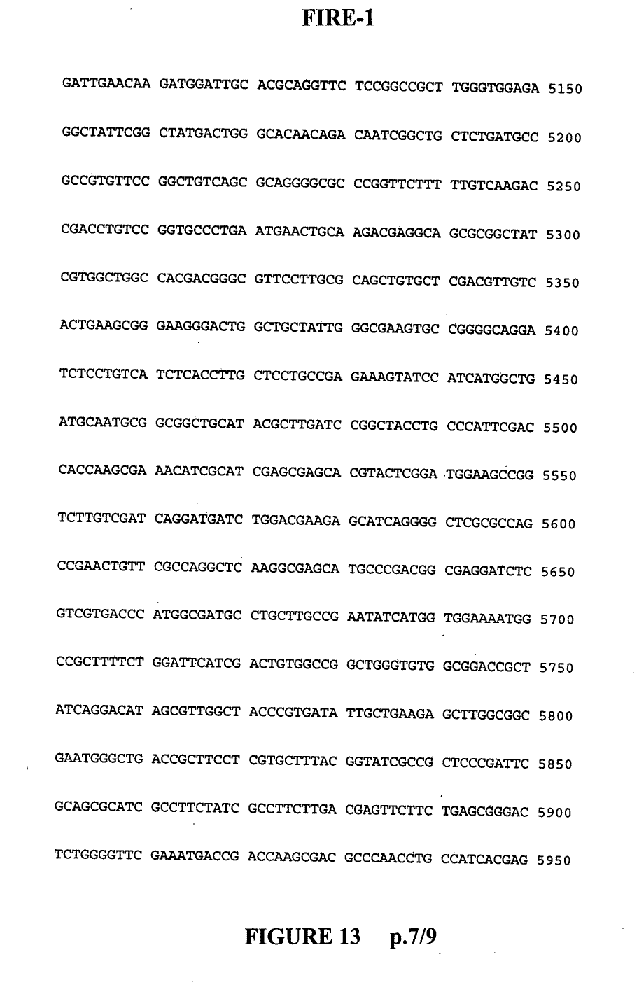

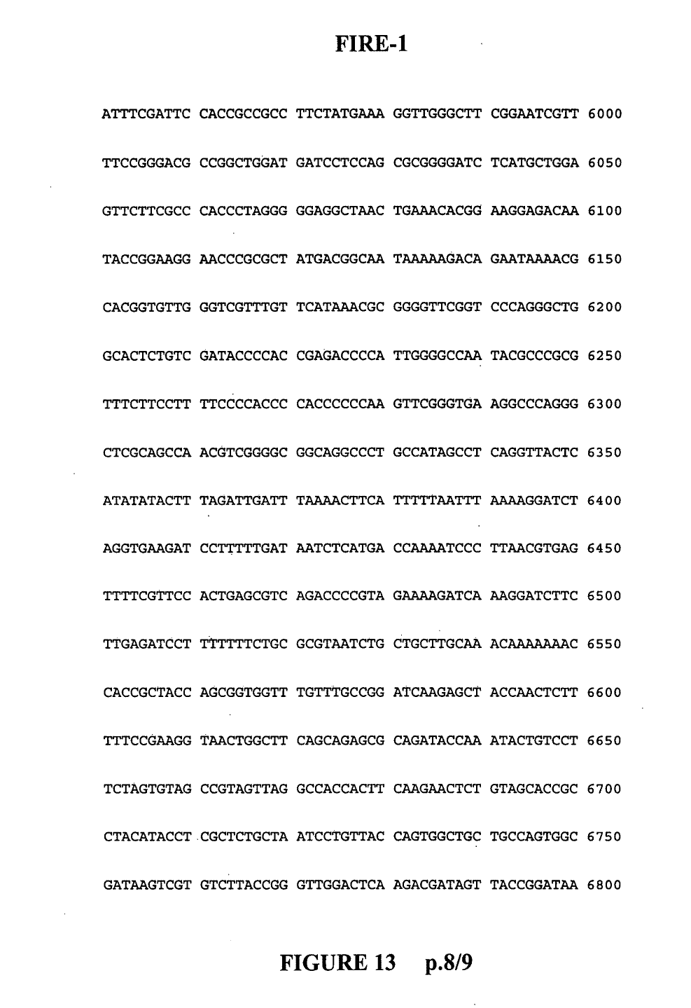

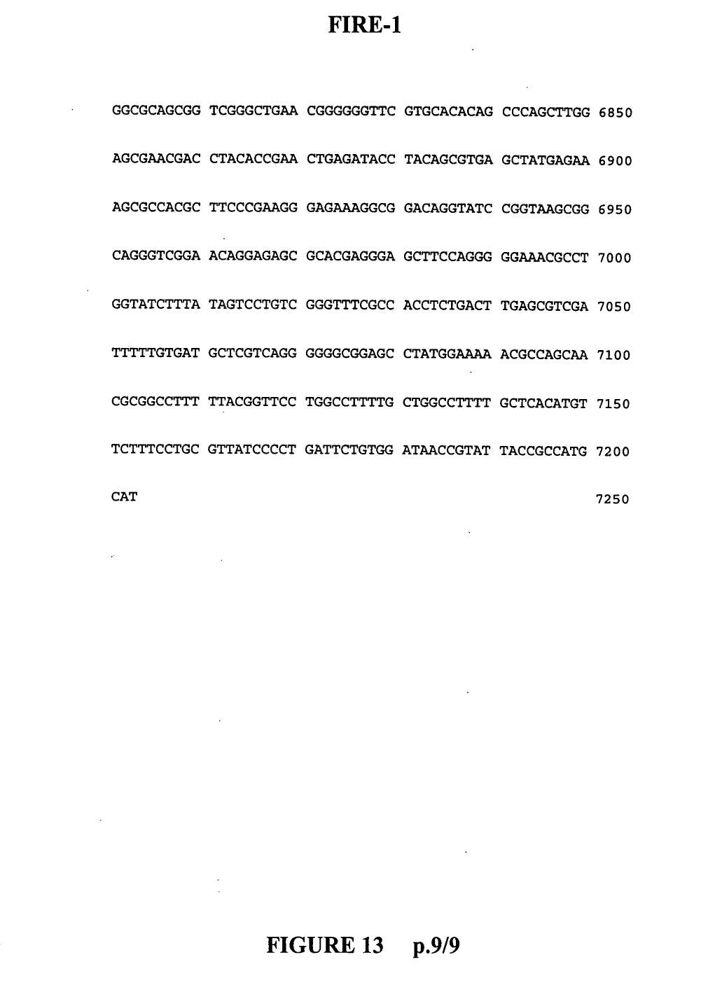

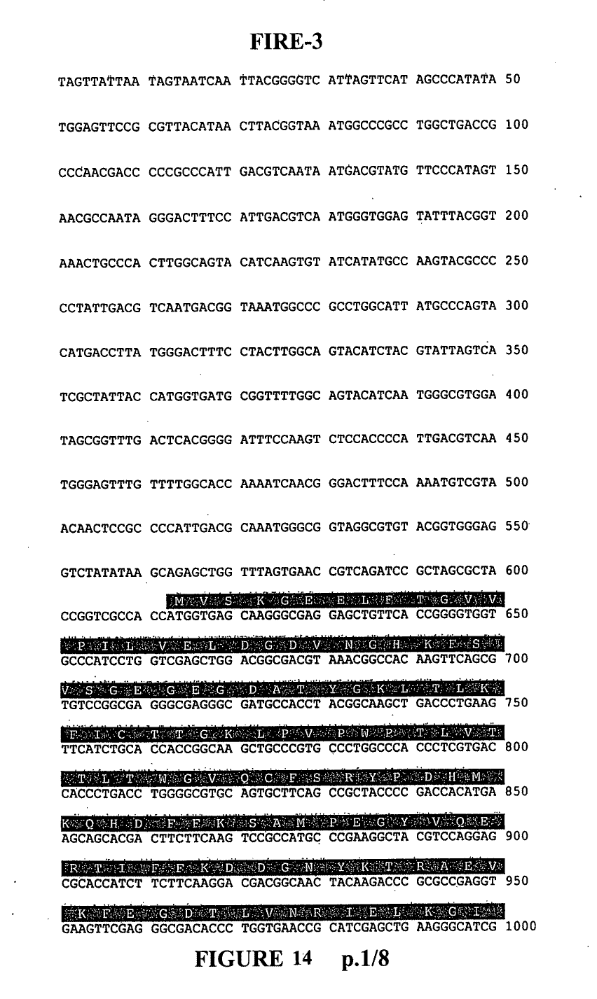

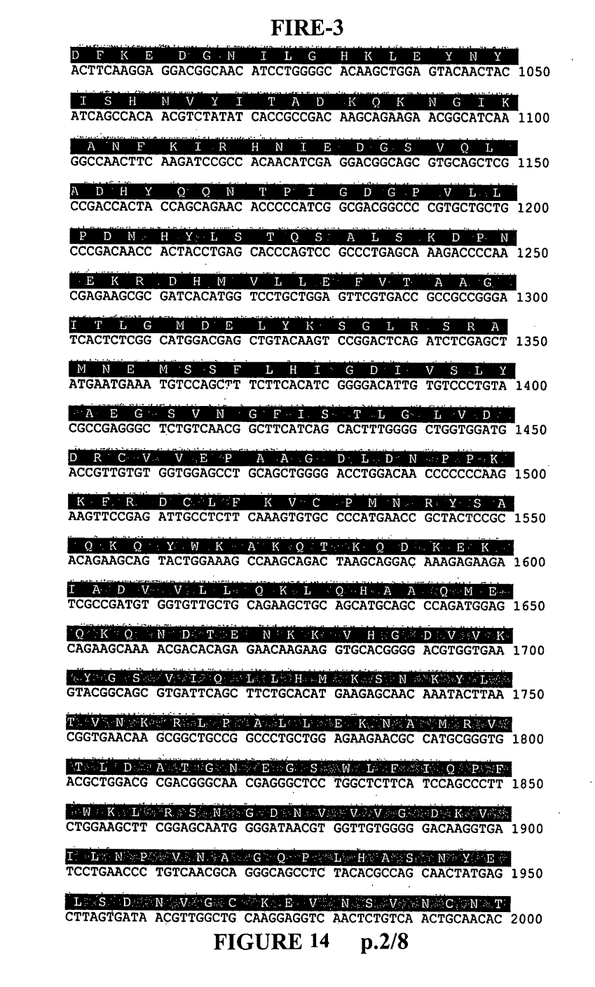

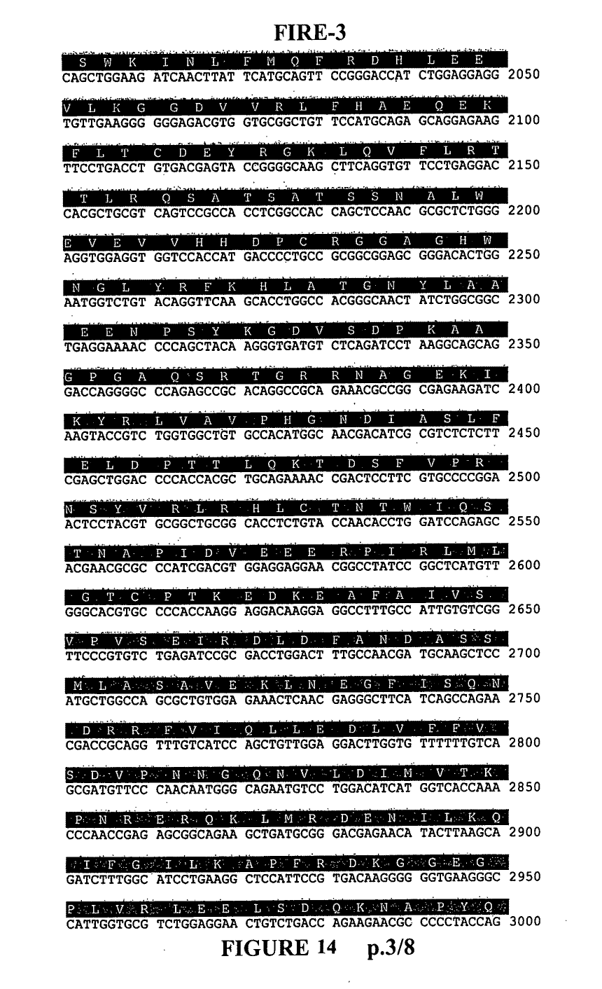

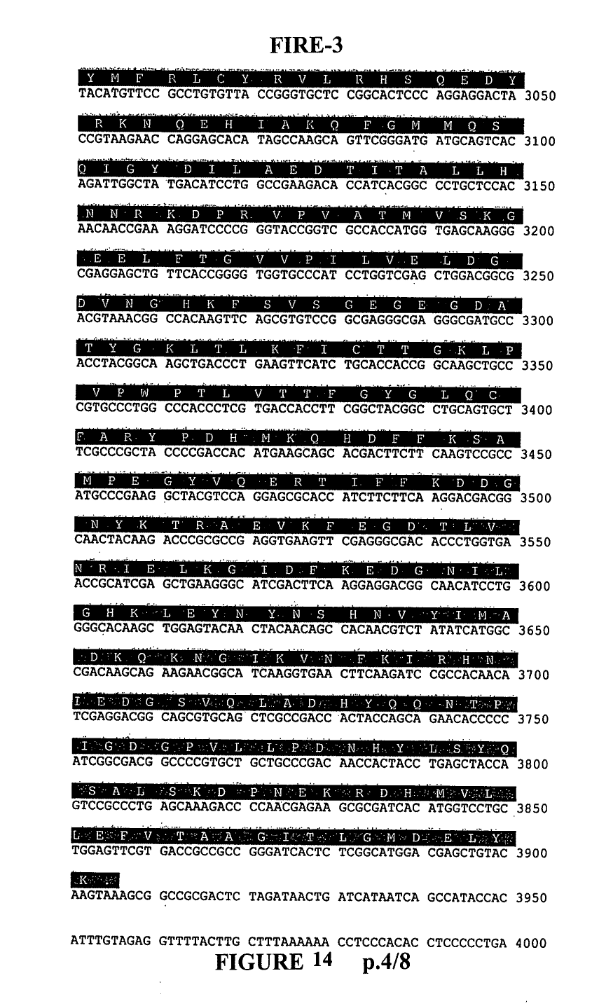

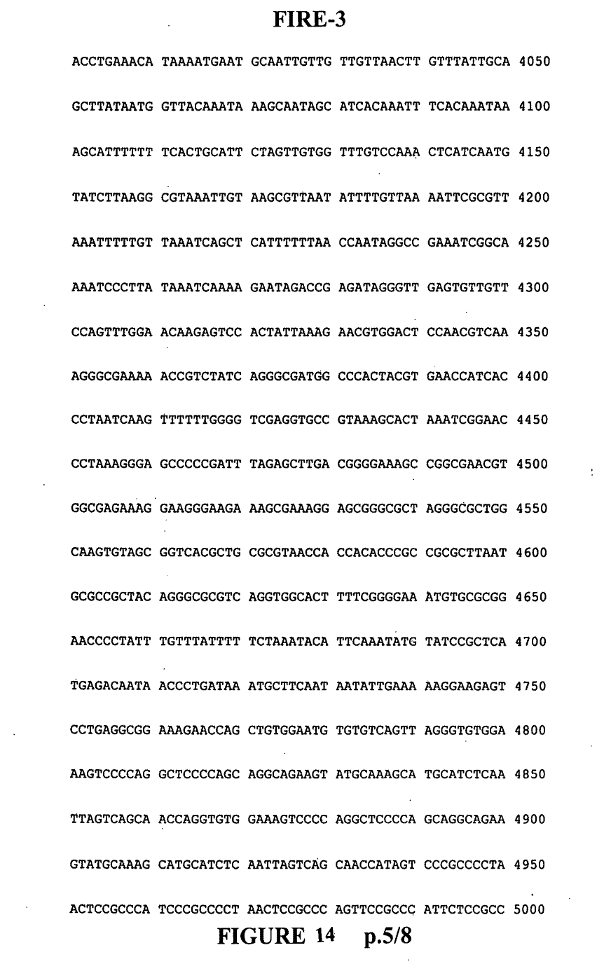

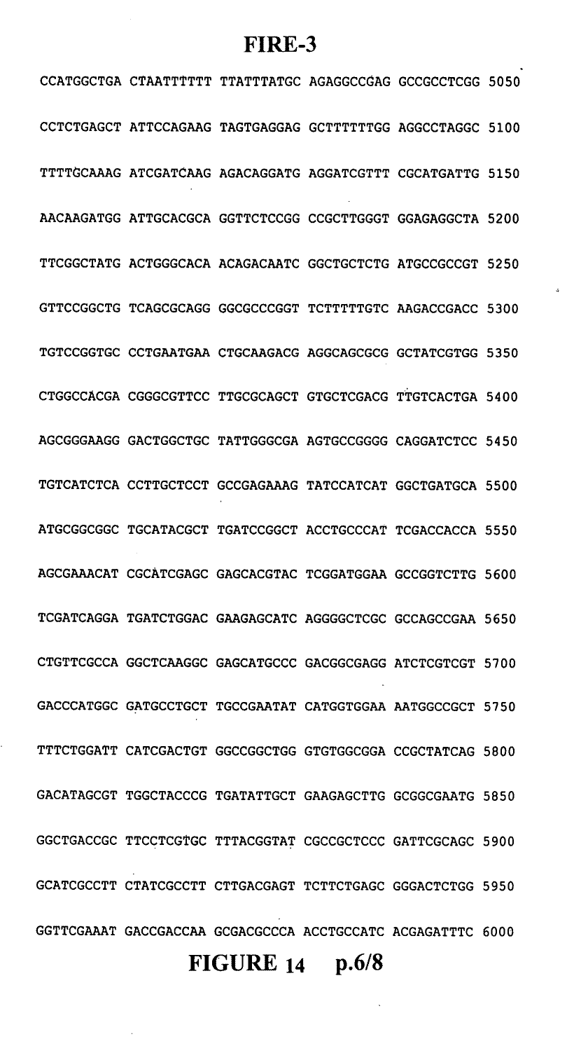

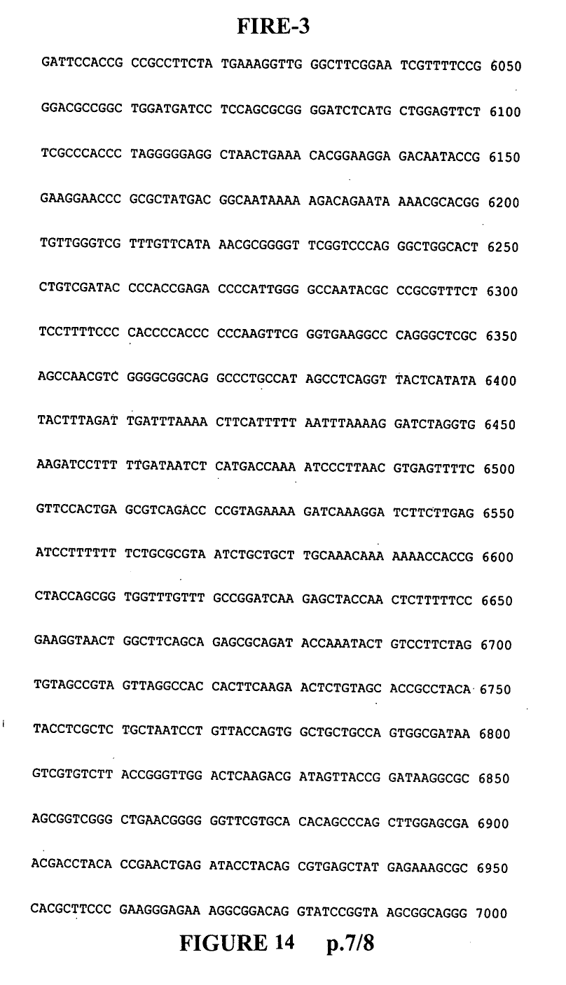

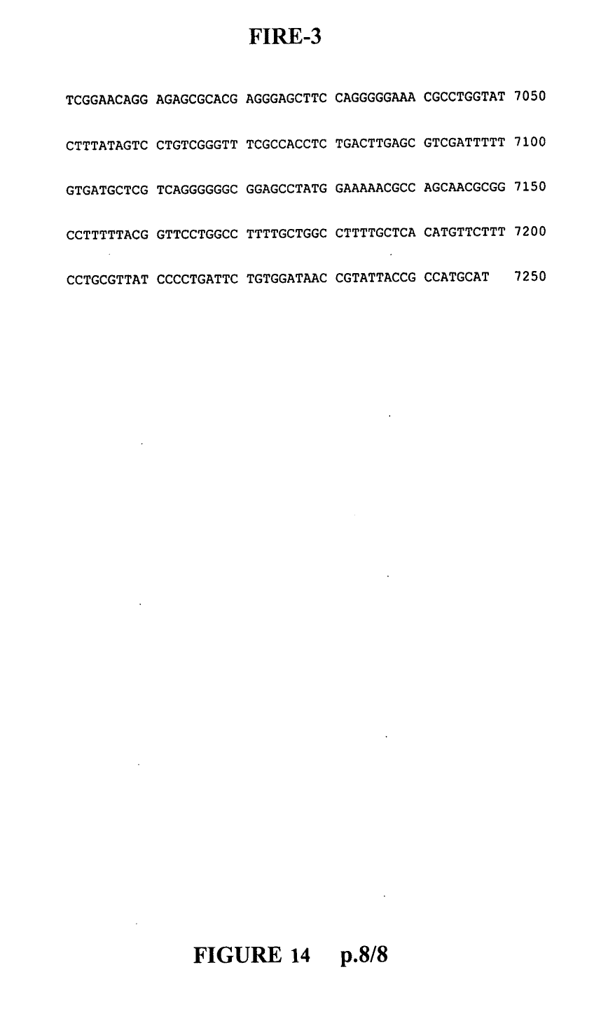

The PCR product from the type-1 receptor ligand binding region was digested with Xho I/Bam HI and ligated into similarly digested pECYFP to form FIRE-1. FIRE-3 was generated by digesting the type-3 derived PCR product with Xho I/Bam HI and first inserting the 1197 nt Xho I-Bam HI fragment into Xho I/Bam HI digested pECYFP followed by the insertion of the 3′ 622 nt Bam HI fragment. FIRE-1 plasmid is shown in FIG. 10, FIRE-3 plasmid is shown in FIG. 11, and the nucleotide assignments of both FIRE plasmid inter-domain boundaries is given in FIG. 12. Nucleotide sequences of FIRE-1 and FIRE-3 are given in FIGS. 13 and 14 respectively.

Construction of FIRE-1 Adenoviral Vector: The FIRE-1 plasmid, described above, was used as the progenitor for the FIRE-1 adenovirus. The adenoviral vector was constructed using a commercially available kit, AdEasy™ XL Adenoviral Vector System (Stratagene, La Jolla, Calif.). The terminally fluorescent tagged InsP₃ ligand binding domain was excised by digesting with Nhe I, Klenow repaired followed by digestion with Not I. This fragment was sub-cloned into the MCS of the shuttle vector (pShuttle-CMV) by digesting the vector with Bgl II and following Klenow repair digested with Nhe I to produce pShuttle-CMV-FIRE-1. The bacterial cell line BJ5183-AD-1, pretransformed with the plasmid pAdEasy-1, was used for in vivo homologous recombination with pShuttle-CMV-FIRE-1. The pAdEasy-1-FIRE-1 insert containing plasmid was transformed into DH5α and produced in bulk. Purified, AdEasy-1-FIRE-1 plasmid was used to transfect/infect bacterial cell line AD-293 for virus amplification. FIRE-1AdV virus was plaque purified, amplified, CsCl gradient purified and stored at −80 deg. C.

COS-1 Cell Transfection: COS-1 cells were transiently transfected with expression plasmids for FIRE-1 and FIRE-3 using a diethylaminoethyl-dextran method as described by Mignery, G. A., Newton, C. L., Archer, B. T., 3rd, and Sudhof, T. C. Journal of Biological Chemistry 265, 12679-12685 (1990).

Neonatal myocyte isolation and transfection: Ventricular neonatal cardiac myoctyes were isolated from 1 to 2 day old Sprague-Dawley rat hearts by enzyme digestion as described by Griffin et al. (Griffin, T. M., Valdez, T. V., and Mesta R. American Journal of Physiology Heart & Circulatory Physiology 287 (2004)). Harvested cells were plated in four-well plates on 1% gelatin coated 25 mm square cover slips (10⁶⁻⁷ cells/well) and allowed to recover for 24-48 hours in plating medium (4 parts DMEM/1 part medium 199, 10% horse serum, 5% fetal bovine serum, 1% antibiotic/antimycotic). After recovery, the media was changed to serum-free, antibiotic-free media and the myocytes were transfected with FIRE-1 plasmid following the method supplied by the manufacturer to transfect a 60mm culture vessel included in the Lipofectamine 2000 (Invitrogen Co.) eukaryotic transfection kit. Cells were incubated (3% CO₂, 37 deg. C.) for 48 hours and the media changed after 24 hours, prior to imaging.

Antibodies: The InsP₃R specific antibodies directed against the amino-termini of the InsP₃R1 and -3 isoforms (T1NH, T3NH) used in this study have been described previously (Ramos-Franco, J., Fill, M., and Mignery, G. A. Biophysical Journal 75, 834-839 (1998); Ramos-Franco, J., Bare, D., Caenepeel, S., Nani, A., Fill, M., and Mignery, G. Biophysical Journal 79, 1388-1399 (2000); Ramos-Franco, J., Caenepeel, S., Fill, M., and Mignery, G. Biophysical Journal 75, 2783-279 (1998).

SDS-Polyacrylamide Gel Electrophoresis and Immunoblotting:

SDS-polyacrylamide gel electrophoresis (“SDS-PAGE”) and western blotting were performed using 7.5% SDS-polyacrylamide gels. Visualization was accomplished using enhanced chemiluminescence reagents obtained from Amersham Life Sciences, Arlington Heights, Ill.

In Vitro Fluorescence Measurement: FIRE-1 and FIRE-3 fluorescence measurements were performed on a Sim Aminco, xenon lamp spectrofluorimeter (SLM Instruments). Monochromator excitation and emission slit widths were set at 4 nm. Excitation light was 415 nm using an excitation monochromator and the dual photon counting emission detectors were set at 480 (F₄₈₀) and 530 nm (F₅₃₀), respectively. Fluorescence measurements were recorded (at 22° C.) in polystyrene cuvettes containing 1 ml of 50 mM Tris-HCl pH 8.3, 1 mM EDTA, and 1 mM phenylmethylsulfonyl fluoride containing 250-300 μg COS-1 cell cytosol fraction expressing FIRE-1 or -3. For inositol phosphate binding experiments, increasing concentrations of inositol-phosphates were added directly to the sample and rapidly mixed prior to fluorescence emission recording. Inositol-phosphate binding affinity (K_{d}) was calculated from changes of Δ(F₅₃₀/F₄₈₀) or percent changes of F₅₃₀/F₄₈₀ as a function of [InsP₃], using non-linear regression analysis conducted with Prizm 4.0 (GraphPad Software, Inc., San Diego Calif.).

Adult Cardiac Myocytes Culture and Adenoviral Infection:

Adult cat ventricular myocytes were isolated, seeded on laminin-coated glass cover slips, and non-adherent cells were removed after 30-45 min. Culture media consisted of serum-free medium 199 (M199) supplemented with (in mM) 25 NaHCO₃, 5 creatine, 5 taurine, 2 carnitine, and 0.1 ascorbic acid. Insulin (100 U/ml), 5′-bromo-2′-deoxyuridine (31 μg/ml), BSA (0.2%) and 2% penicillin-streptomycin were also added to the media. Myocytes were then exposed to recombinant replication-deficient adenovirus expressing the FIRE-1 sensor for 2 hr at an multiplicity if infection (“MOI”) of 1-10. Myocytes were subsequently cultured for 24-36 hrs and media was changed twice daily.

Confocal Microscopy and Patch Clamping:

A cover slip with cells expressing the FIRE probe was positioned to the stage of an inverted microscope equipped with an x40 1.3 NA oil immersion objective lens. Cells were continuously superfused with Tyrode solution (140 mM NaCl, 5 mM KCl, 2 mM CaCl₂, 1 mM MgCl₂, 10 mM glucose, and 10 mM Hepes; pH 7.4 (adjusted with NaOH)). Changes in FRET were measured with laser scanning confocal microscopy (Radiance 2000 MP, Bio-Rad, UK). CFP was excited with the 457 nm line of an argon ion laser. CFP and YFP emissions were measured at wavelengths 488 (F₄₈₈) and >530 nm (F₅₃₀), respectively. Changes in InsP₃ activity were defined as the relative change in the ratio F₅₃₀/F₄₈₈ of the background corrected fluorescence intensities measured at the emission wavelengths of CFP and YFP. Experiments were conducted at room temperature.

In experiments where myocytes were dialyzed with InsP₃ (10 μM) the conventional patch-clamp technique was used. Microelectrodes had resistances of 2-4 MΩ when filled with an intracellular solution containing 100 mM potassium glutamate, 40 mM KCl, 1 mM MgCl₂, 4 mM Na₂ATP, 10 mM HEPES, 0.1 mM EGTA, pH 7.2 (adjusted with KOH). Myocytes were voltage-clamped at a holding potential of −70 mV.

For intracellular Ca measurements, ([Ca]ᵢ) cells were loaded for 20 minutes with the membrane permeant fluorescent Ca indicator fluo-4/AM (Molecular Probes/Invitrogen; 20 μM). Fluo-4 was excited with the 488 nm line of an argon ion laser and emitted Ca-dependent fluorescence was measured at wavelengths >515 nm. [Ca]ᵢ signals are presented as background-subtracted normalized fluorescence (F/F₀) where F is the fluorescence intensity and F₀ is resting fluorescence recorded under steady-state conditions at the beginning of an experiment.

### Results

Construction and Expression of FRET-Based Biosensor FIRE:

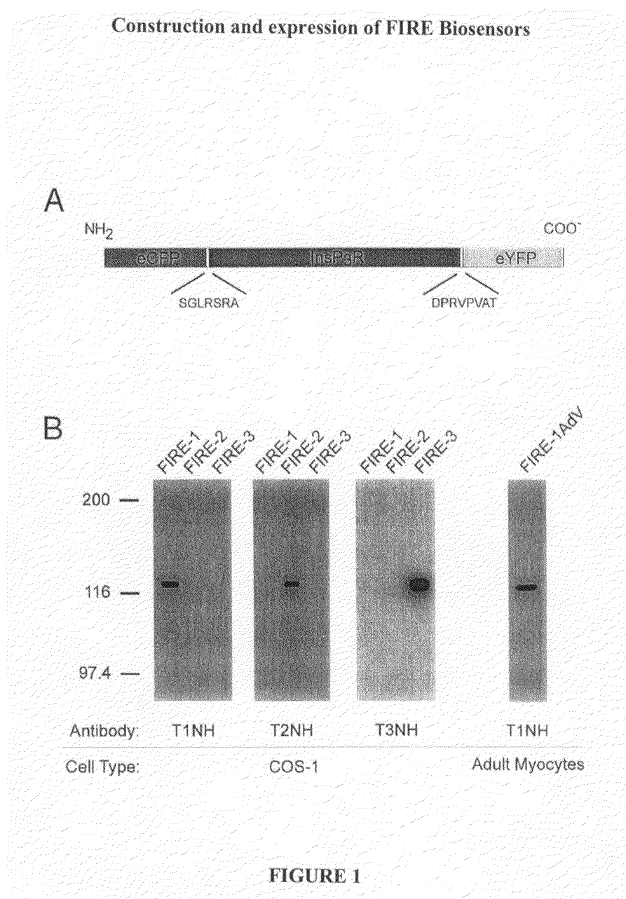

A set of fluorescent reporter-ligand binding domain chimeras from the type-1 and -3 InsP₃R isoforms terminally fused with CFP and YFP were constructed. The constructs span the receptors ligand binding core encompassing the amino-terminal 589 residues of the type-1 (SI⁻) spliced form and 604 amino acids for type-3 homologue linked amino-terminally with CFP and carboxyl-terminally with YFP. In all cases minimal linker sequences (7 residues CFP-InsP₃R and 8 residues for InsP₃R-YFP junctions) were used to join the fluorescent proteins to the InsP₃R binding core backbone (see FIG. 1A). These are the FIRE plasmids. The InsP₃R isoforms are identified as FIRE-1 and FIRE-3.

COS-1 cells were transiently transfected with the FIRE-1 and FIRE-3 plasmids and soluble fractions were examined by western immunoblotting using InsP₃R amino-terminal antibodies specific for the type-1 or -3 InsP₃R. In addition, FIRE-1 was also introduced into adult ventricular myocytes using an adenoviral expression system (FIRE-1Adv). As shown in FIG. 1B, all constructs express at high levels as soluble proteins with an expected Mᵣ ˜118 kD.

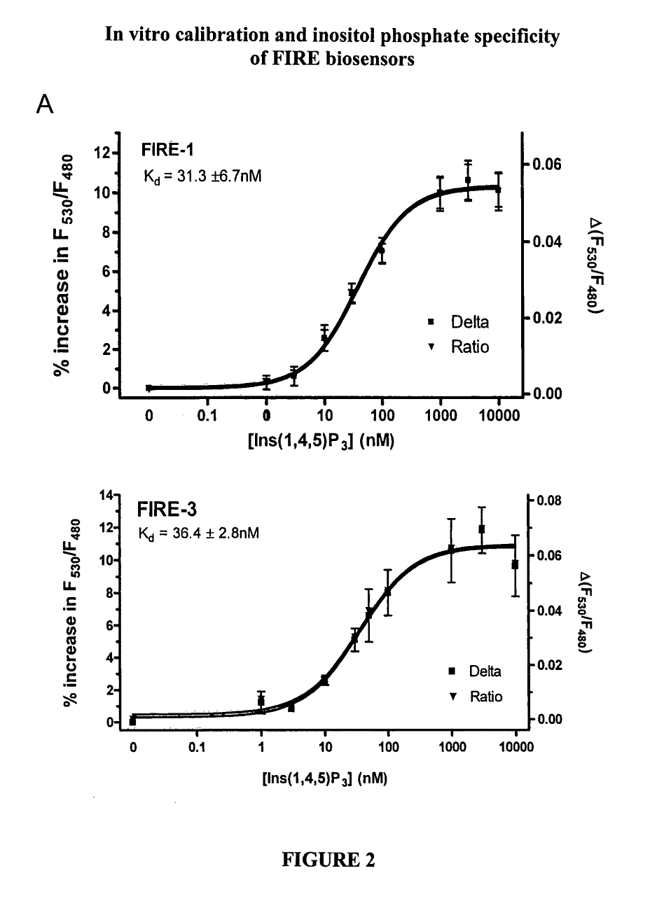

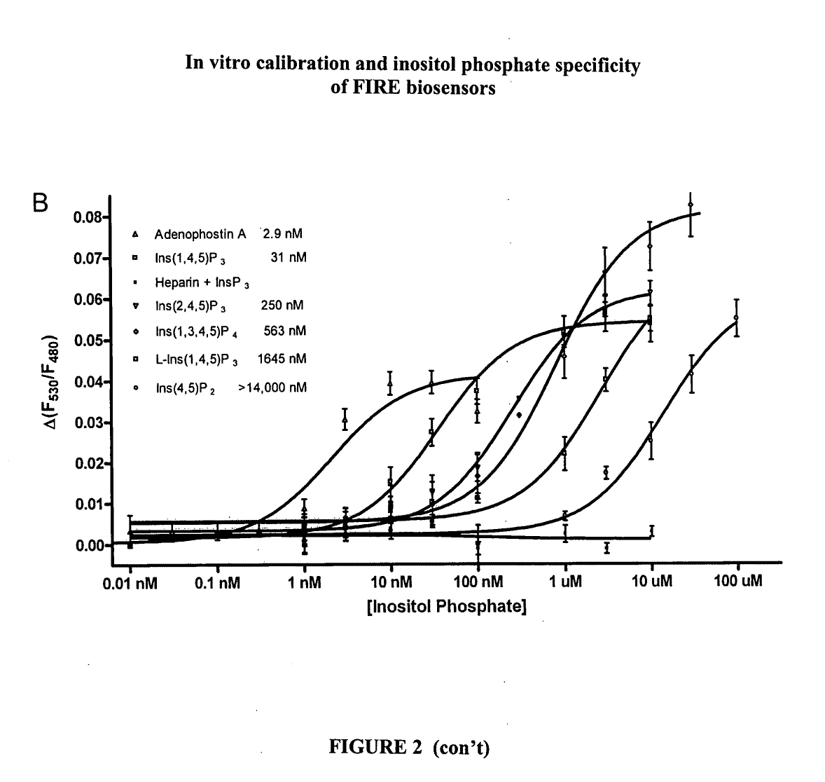

Calibrations and Selectivity: FIG. 2A shows the [InsP₃]-dependence of the FRET signal (expressed as change in F₅₃₀/F₄₈₀) for both FIRE-1 and FIRE-3 expressed in COS-1 cells. Cytosolic extracts from FIRE expressing COS-1 cells were suspended in cuvettes and placed in a fluorimeter. Incremental addition of InsP₃ (0-10 μM) resulted in enhanced FRET over a range from 1 nM to 1 μM InsP₃. Both FIRE-1 and FIRE-3 sensors exhibited ˜11% change in the fluorescence ratio with apparent K_{d} of 31.3±6.7 nM (n=7) and 36.4±2.8 nM (n=4) respectively.

The specificity and selectivity of FIRE-1 to InsP₃ (Ins(1,4,5)P₃) was further examined by characterizing the response to other inositol-phosphates, an InsP₃R-agonist, and heparin (FIG. 2B). Preincubation of the COS cytosol with 5 mg/ml heparin, followed by addition of InsP₃, resulted in essentially no change in FRET, confirming that the InsP₃ induced change in fluorescent ratio is a consequence of InsP₃ binding to the FIRE-1 ligand binding region. Adenophostin A, a high affinity InsP₃R agonist, showed nearly 11× higher potency (K_{d}=2.9 nM, n=4−8) and slightly less efficacy at increasing FRET (76% of the maximal ΔF₅₃₀/F₄₈₀ measured for Ins(1,4,5)P₃). Of the inositol phosphates examined, Ins(2,4,5)P₃, which is often used as a more stable, poorly metabolized agonist of the InsP₃R, exhibited ˜8 fold lower apparent affinity (250 nM, n=4) for FIRE-1 than did Ins(1,4,5)P₃. Ins(1,3,4,5)P₄ and Ins(4,5)P₂ induced changes in ΔF₅₃₀/F₄₈₀ with apparent affinities of 563 nM (n=3) and >14 μM, (n=4) respectively. The primary cellular degradation product of Ins(1,4,5)P₃ through the activity of InsP₃-5 phosphatase (Shears, S. B. Advances in Second Messenger & Phosphoprotein Research 26, 63-92 (1992)), Ins(1,4)P₂, did not increase FRET at any concentrations up to 100 μM (n=4, not shown). L-myo-Ins(1,4,5)P₃ induced FRET in FIRE-1 with significantly lower potency (n=4) than the biologically relevant D-myo stereoisomer, consistent with previous reports regarding the stereo specificity of the InsP₃R (Nahorski, S. R., Potter, B. V. L. Trends in Pharmacological Sciences 10, 139-144 (1989); Wilcox, R. A., Primrose, W. U., Nahorski, S. R., and Challiss, R. A. Trends in Pharmacological Sciences 19, 467-475 (1998)).

The relative potency of these inositol phosphates are very similar to those observed in competition binding assays using NH-terminal fragments encompassing the ligand binding domain of the type-1 and type-2 InsP₃R. Additionally, the interaction of these inositol phosphates with the ligand binding region of the FIRE-1 expression product reflects the relative potency of the inositol phosphates in their ability to release Ca from InsP₃ sensitive stores in cultured cells (SH-SY5Yand Swiss-3T3), as shown by Wilcox, R. A., Challiss, R. A., Liu, C., Potter, B. V., and Nahorski, S. R. Molecular Pharmacology 44, 810-817 (1993); Willcocks, A. L., Strupish, J., Irvine, R. F., and Nahorski, S. R. Biochemical Journal 257, 297-300 (1989). In those studies, Ins(2,4,5)P₃ and Ins(1,3,4,5)P₄ were ˜10× and ˜18× less potent than Ins(1,4,5)P₃ in releasing Ca. Thus, FIRE-1 is an excellent biosensor for signals that are expected to activate the InsP₃R.

Detection of [InsP₃] in COS-1 Cells: The response of FIRE-1 and FIRE-3 transfected COS-1 cells to InsP₃ liberating agonists were examined using confocal microscopy. As seen in FIG. 3A, stimulation of COS-1 cells with 10 μM ATP reveals oscillatory increases in [Ca]; that are mediated by InsP₃-dependent Ca release. In parallel experiments, cells expressing FIRE-1 and FIRE-3 were stimulated with 10 μM ATP (FIG. 3B) resulting in a similar oscillatory increase in FRET assessed by the F₅₃₀/F₄₈₈ ratio with excitation at 457 nm. FIG. 3C shows that the maximal increase of 5-6% in the F₅₃₀/F₄₈₈ ratio induced by ATP or acetylcholine would correspond to free [InsP₃] in the range of 20-50 nM based on the calibrations in FIG. 2A.

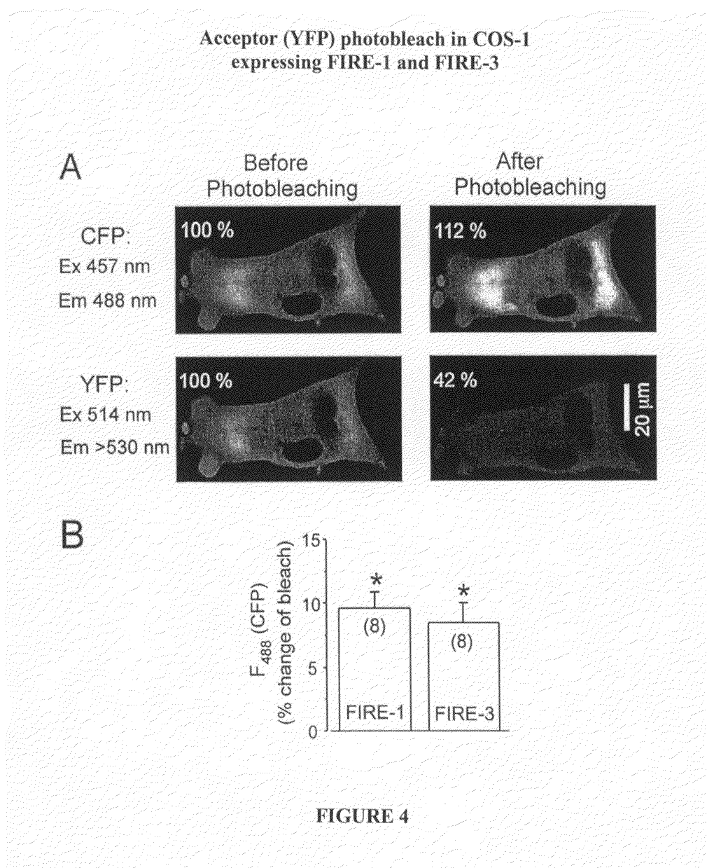

A critical hallmark of FRET is an increase in donor (CFP) fluorescence upon bleaching of the acceptor (YFP). FIG. 4 illustrates that partial photo-bleach of YFP (with the 514 nm laser line; there is a 58% decrease in YFP fluorescence) increases CFP donor fluorescence by 12% in a COS-1 cell expressing FIRE-1. Mean values for increased CFP fluorescence after YFP photobleach are 8.9±1.3% (n=8) and 8.5±1.5% (n=8) for FIRE-1 and FIRE-3, respectively. These are lower limits for the amount of basal FRET (before [InsP₃] increases), because the mean extent of YFP photobleach was only 58.2±3.6% (n=8) and 54.9±4.6% (n=8) respectively. Correcting for incomplete YFP photobleach, we estimate that the basal extent of FRET is about 22.6±2.1% and 17.2±2.2% for FIRE-1 and -3 respectively.

Detection of [InsP₃] in Cardiac Myocytes: The FIRE-1 plasmid was transfected into cultured neonatal rat ventricular myocytes and examined if the cells exhibited altered FRET upon stimulation with endothelin-1 (ET-1). Although the number of cells transfected was low, the cells expressing FIRE-1 were readily identifiable by their fluorescence, which as in COS-1 cells was cytosolic. Exposure of these neonatal myocytes to 100 nM ET-1 resulted in increased FRET indicating InsP₃ generation (FIG. 5). Compared to untreated controls, 100 nM ET-1 induced an 8% peak increase in the F₅₃₀/F₄₈₈ ratio (n=4; FIG. 5). The InsP₃ induced increase in FRET was detected within 20-25 seconds after addition of ET-1 and reached a maximum within 1-2 min. In the continued presence of ET-1 the F₅₃₀/F₄₈₈ ratio leveled off to a sustained plateau, suggesting maintained levels of elevated [InsP₃]. Removal of the agonist resulted in a decline of the signal to baseline levels within 1 minute. Similar to our photobleaching experiments in COS-1 cells, FIRE-1 expressed in the neonatal ventricular myocytes also shows comparably increased CFP fluorescence (˜11%) after YFP photobleach.

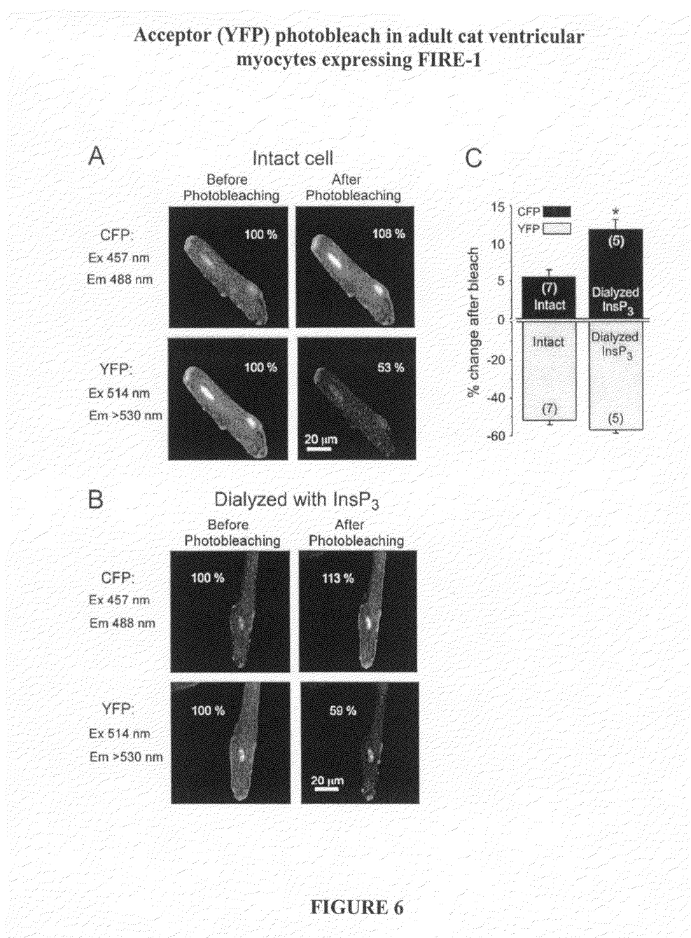

Adult ventricular myocytes are a main focus of cardiac research, although work on cultured neonatal rat ventricular myocytes has been used extensively for studying signaling pathways. Adult ventricular myocytes cannot be transfected by plasmids. Therefore the FIRE-1 coding region was excised from the plasmid vector and introduced into the AdEasy XL adenoviral vector system to produce FIRE-1AdV. Infection of adult cat ventricular myocytes resulted in the expression of a protein of Mᵣ ˜118K that immuno-reacts with our type-1 specific amino terminal antibody (T1NH) (FIG. 1B, right panel). This expression product is soluble and essentially indistinguishable from the plasmid based FIRE-1 expression products. Unlike COS-1 or neonatal myocytes, expression of FIRE-1 in adult cells revealed some sequestration of the sensor to the nuclei (FIG. 6A). Whether this apparent nuclear enrichment results from fluorescent protein over-expression, or another feature unique to expression of this chimera in this cell type is unknown.

In FIRE-1 expressing adult myocytes, evaluation of increased CFP fluorescence upon YFP photobleach, with or without saturating amounts of InsP₃ (FIG. 6) was done. As observed in COS-1 cells, YFP photobleach (by 47% in the example shown in FIG. 6A) resulted in an increased CFP fluorescence by 8%. To elevate [InsP₃] and saturate FIRE-1, adult myocytes were patch-clamped with pipettes containing 10 μM InsP₃, such that upon rupture of the patch InsP₃ diffused into the cell (FIG. 6B; see also below). In this cell dialyzed with InsP₃, a 41% bleach of YFP resulted in increase of CFP fluorescence by 13%. Mean data from these experiments as seen in FIG. 6C show that for a comparable extent of YFP photobleach (51.9±2.3%; n=7 and 57.0±1.6%; n=5, respectively), there was considerably greater increase of CFP fluorescence at high [InsP₃]ᵢ (11.8±1.2% vs. 5.4±1.0%). This confirms that increased [InsP₃] augments FRET in FIRE-1 in-vivo in adult cardiac myocytes.

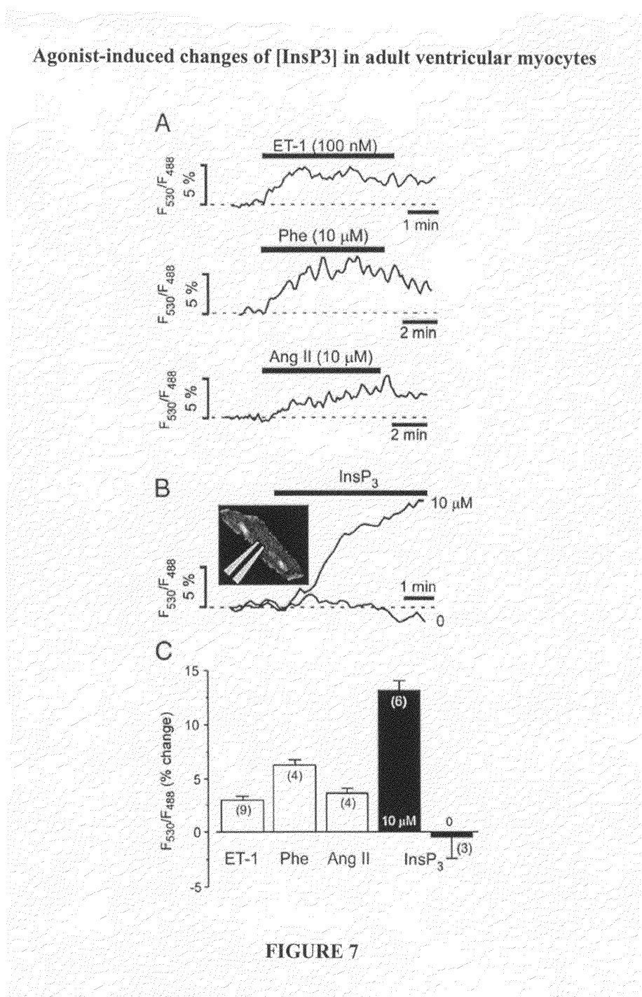

FIG. 7A shows the influence of three GPCR agonists (ET-1; phenyl-ephrine (“Phe”); angiotensin II, (“Ang II”)) to induce increases in [InsP₃]; in adult cat ventricular myocytes expressing FIRE-1. All three agonists induced rapid and readily detectable increases in free [InsP₃]; in adult cardiomyocytes. ET-1 tended to produce the fastest increases (see time bars), but Phe at 10 μM produced the largest increase in [InsP₃]ᵢ. To assess the dynamic range of FIRE-1 in these adult cardiac myocytes the cells were dialyzed (via patch pipette) with either InsP₃-free solution or 10 μM InsP₃ (FIG. 7B). Having no InsP₃ in the pipette did not significantly affect the F₅₃₀/F₄₈₈ ratio, suggesting that resting [InsP₃] may be low. In contrast, internal perfusion with 10 μM InsP₃ increased the ratio in cells by 13.2±0.9% (n=6), which is comparable to our in vitro calibrations in FIG. 2. Assuming the K_{d} from FIG. 2, this would correspond to increases of [InsP₃]; in the range of 10-30 nM with these three agonists in adult ventricular myocytes. FIG. 7C summarizes the average changes in InsP₃ (F₅₃₀/F₄₈₈) elicited with GPRC agonists and InsP₃ perfusion, respectively, in adult ventricular myocytes. These results demonstrate the ability of FIRE-based FRET sensors to detect temporal changes in [InsP₃] in isolated cells and underscore the utility of this indicator to measure InsP₃ levels in real time.

Spatially resolved [InsP₃]ᵢ signals: FIRE-1 can also provide spatially resolved information concerning InsP₃ signaling. FIG. 8A shows (top) the spatial distribution of FIRE-1 in an adult cat ventricular myocyte (excitation 457 nm; emission >530 nm). In this experiment, the FIRE-1 expressing myocyte was internally dialyzed with InsP₃ via a patch pipette containing 10 μM InsP₃. The line trace in FIG. 8A shows the longitudinal profile of F₅₃₀/F₄₈₈ 5 min after patch rupture. As expected, [InsP₃] was highest directly beneath the pipette and declined as a function of distance from the InsP₃ source. FIG. 8B shows the time course of changes of [InsP₃] in four regions of interest (“ROI”):1) under the pipette, 2) in a nearby nucleus, 3) cytosol just beyond the nucleus, and 4) distant cytosol, as a function of time. Clearly the rise in [InsP₃]; is slower in more distant regions, and notably the rise in the nearby nucleus (2) is no faster than a more distant cytosolic ROI 3. This indicates that the nuclear envelope slightly retards InsP₃ diffusion. The inset in FIG. 8B plots the half-time (t_{1/2}) of rise of local [InsP₃]ᵢ as a function of distance from the pipette (2 experiments). The t_{1/2} increases roughly linearly as a function of distance in the cytosol (open symbols), whereas InsP₃ diffusion into the nucleus was substantially delayed (filled symbols) compared to the cytosol. This further indicates slower InsP₃ diffusion into the nucleus than along the cytosol.

A more physiological stimulus to explore spatio-temporal differences in [InsP₃]ᵢ between nucleus and cytosol was used. FIG. 9 shows an intact adult ventricular myocyte expressing FIRE-1 upon exposure to 100 nM ET-1. ET-1 caused [InsP₃] to rise more rapidly and to higher levels in the cytoplasm as compared to the nucleus (FIG. 9A). Average data indicates that the amplitude of rise in nuclear [InsP₃] was 70±9% (n=6) of that in the cytosol and the t_{1/2} was about twice as long (FIG. 9B). The data from FIGS. 8 and 9 suggest that InsP₃ can diffuse over long distances in the cytosol, that the nuclear pores slows down this diffusion into nuclei, and that neurohumoral activation of InsP₃ in intact ventricular myocytes causes [InsP₃] to rise rapidly in both cytosol and nucleus.

In addition to sensing intracellular changes of InsP₃ with temporal and spatial resolution, the FIRE expression products are functional in vitro and respond to incremental additions of InsP₃ (see FIG. 2). This property enables a dual use for these recombinant proteins in that FIRE expression products can be used in a rapid in vitro fluorometric assay to measure samples containing unknown concentrations of InsP₃.

Historically, measurement of InsP₃ concentrations in cellular extracts or homogenates have involved competition radio-ligand binding assays. In essence, competition radio-ligand binding assays involve the measurement of a radioactively labeled molecules (e.g., InsP₃) ability to bind to a substrate, which is usually InsP₃R protein from cerebellum or adrenal gland. A fixed amount of radiolabeled InsP₃ is co-incubated with increasing concentrations of unlabeled InsP₃ together with the InsP₃R and the amount of radioactive InsP₃ bound is determined by scintillation counting. This is then used as a standard curve from which radiolabeled InsP₃ binding assays in the presence of extracts containing unknown concentrations of InsP₃ can compared. From these, the investigator can determine the InsP₃ concentration from their unknown sample. This form of assay for InsP₃ could be performed in the laboratory using the appropriate reagents and until recently was available as a kit from Amersham. There are, however, several disadvantages to competition binding methodologies. The primary drawback is the use of radioactive material which is environmentally dangerous, costly to synthesize and poses storage and disposal problems.

Alternatively, the use of FIRE-based fluorescence measurements to quantitate InsP₃ requires no radioactivity or toxic scintillation fluids for quantification. Tissue extracts or cellular homogenates containing unknown concentrations of InsP₃ could be rapidly analyzed using FIRE. The InsP₃ concentration of these extracts could be determined by measuring the FRET induced to FIRE in the fluorometric assay by incremental addition of the unknown sample. These values can be converted directly to InsP₃ concentration by extrapolation from a FIRE calibration curve in which known amounts of InsP₃ were added, as in FIG. 2. Therefore, it is envisioned that an in vitro test kit comprising the affinity purified FIRE expression products, reagents, and standards, can be used for the quantification of InsP₃ and will result in enhanced sensitivities and dynamic ranges.

As used herein, an amino acid sequence or a nucleotide sequence is “substantially identical” to a reference sequence if the amino acid sequence or nucleotide sequence has at least 90% sequence identity (e.g., 90% or greater) with the reference sequence over a given comparison window. As used herein, an amino acid sequence or a nucleotide sequence is “substantially similar” to a reference sequence if the amino acid or nucleotide sequence has at least 80% (e.g., 80% or greater) with the reference sequence over a given comparison window. Sequence identity is calculated based on a reference sequence.

Additionally, degenerate variants of the nucleic acids that encode the proteins of the present invention are also provided. Degenerate variants of nucleic acids comprise replacement of the codons of the nucleic acid with other codons encoding the same amino acids. In particular, degenerate variants of the nucleic acids are generated to increase its expression in a host cell.

All publications and patents cited in this specification are hereby incorporated by reference herein as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The Sequence Listing (paper) of this specification incorporates by reference the computer readable disc also enclosed with this patent application.

We claim:

1. A nucleotide sequence encoding a genetically engineered biosensor protein comprising a binding ligand of InsP₃ and one fluorescent molecule on the amino terminus and a different fluorescent molecule on the carboxyl terminus, having a nucleotide sequence of SEQ ID NO 1.

2. The nucleotide sequence of claim 1, wherein said nucleotide sequence is substantially identical to SEQ ID NO 1.

3. The nucleotide sequence of claim 1, wherein said nucleotide sequence is substantially similar to SEQ ID NO 1.

4. A nucleotide sequence encoding a genetically engineered protein comprising a binding ligand of InsP₃ and a one fluorescent molecule on the amino terminus and a different fluorescent molecule on the carboxyl terminus, having a nucleotide sequence of SEQ ID NO 2.

5. The nucleotide sequence of claim 4, wherein said nucleotide sequence is substantially identical to SEQ ID NO 2.

6. The nucleotide sequence of claim 4, wherein said nucleotide sequence is substantially similar to SEQ ID NO 2.

7. A genetically engineered protein comprising an amino acid sequence of a binding ligand having two termini, each terminus having a different fluorescent molecule whereby upon coming into proximity with each other exhibit fluorescence resonance energy transfer, wherein said amino acid sequence is substantially identical to the sequence selected from the group consisting of SEQ ID NO 6 and SEQ ID NO 7.

8. A genetically engineered protein of claim 7, wherein said amino acid sequence is substantially similar to SEQ ID NO 6.

9. A genetically engineered protein of claim 7, wherein said amino acid sequence is substantially similar to SEQ ID NO 7.

10. A vector comprising the nucleotide sequence of claim 1.

11. A vector comprising the nucleotide sequence of claim 4.

12. A kit comprising at least one protein according to claim 7.


SEQUENCE LISTING (7 sequences)

SEQ ID NO: 1 - DNA, 7203 bp, Artificial
  plasmid InsP3 isoform 1
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctatgtctgacaaaatgtctagtttcctacatatcggagacatttgttctctgtatgcagagggatctacgaatggatttatcagcaccttgggcttggttgatgaccgttgcgttgtacagccagaagctggggaccttaacaatccacccaagaaattcagagactgcctctttaagctatgtcctatgaatcgatattctgcacagaagcagttctggaaagctgctaagcccggggccaacagtacaacagatgcggtgctgctcaacaaattgcatcatgctgcagacttggaaaagaagcagaatgagacagaaaacaggaaattgctggggacggtcatccagtatggcaacgtgatccagctcctacatttgaaaagcaataaatacttaactgtgaataagaggcttcctgccctactggagaagaacgccatgagagtgacgttggatgaggctggaaatgaagggtcttggttttacattcagccgttctacaagctccgctccatcggagacagtgtggtcataggtgacaaggtagttttgaaccctgtcaatgctgggcagccgctacatgccagcagccatcagctggtggataacccgggctgcaatgaggtcaactccgtcaactgtaacacaagctggaaaatagtgcttttcatgaaatggagtgataacaaagatgacattctaaaaggaggtgatgtggtaagactcttccatgctgagcaagagaagtttctcacgtgcgatgagcacaggaagaagcagcacgtcttcctgaggacaaccggcagacagtcagccacgtcggccaccagctccaaagccctgtgggaagtggaggtagtccagcatgacccatgtcggggtggagctgggtactggaatagcctcttccggttcaagcacctggctacagggcattacttggcagcagaggtggaccctgatcaggacgcatctcggagcaggttgagaaatgcccaagaaaaaatggtgtactctctggtctctgtgcctgagggcaacgacatctcctccatctttgagctagaccccacaactctacgtggaggtgacagccttgtcccaaggaactcctatgtccggctcagacacctgtgtacaaacacctgggttcacagcacaaacatccccattgacaaggaagaggaaaaacctgtcatgctgaagattggtacctctcccctgaaggaggacaaggaagcttttgccatagtccccgtttctcctgctgaggttcgggacctggactttgccaatgatgccagcaaggtgctgggctccatcgctgggaagttggaaaagggcaccatcacccagaatgagagaaggtctgtcaccaagctgctggaagacttggtttactttgtcacgggtggaacgaactctggccaagacgtgcttgaagtagtcttctctaagcccaaccgagagcggcagaagctgatgagggaacagaatattctcaagcagatcttcaagcttttgcaggcccccttcacggactgcggggatggcccaatgcttcggctggaggagctgggggaccagcggcacgctcctttcagacacatttgccgactctgctacagggtcctgagacactcacagcaagactacaggaagaaccaggagtacatagccaagcagtttggcttcatgcagaagcagattggctatgatgtgctggccgaagacaccatcactgccctgctccacaacaaccgaaaggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgcgactctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 2 - DNA, 7248 bp, Artificial
  plasmid InsP3 isoform 3
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctatgaatgaaatgtccagctttcttcacatcggggacattgtgtccctgtacgccgagggctctgtcaacggcttcatcagcactttggggctggtggatgaccgttgtgtggtggagcctgcagctggggacctggacaacccccccaagaagttccgagattgcctcttcaaagtgtgccccatgaaccgctactccgcacagaagcagtactggaaagccaagcagactaagcaggacaaagagaagatcgccgatgtggtgttgctgcagaagctgcagcatgcagcccagatggagcagaagcaaaacgacacagagaacaagaaggtgcacggggacgtggtgaagtacggcagcgtgattcagcttctgcacatgaagagcaacaaatacttaacggtgaacaagcggctgccggccctgctggagaagaacgccatgcgggtgacgctggacgcgacgggcaacgagggctcctggctcttcatccagcccttctggaagcttcggagcaatggggataacgtggttgtgggggacaaggtgatcctgaaccctgtcaacgcagggcagcctctacacgccagcaactatgagcttagtgataacgttggctgcaaggaggtcaactctgtcaactgcaacaccagctggaagatcaacttattcatgcagttccgggaccatctggaggaggtgttgaaggggggagacgtggtgcggctgttccatgcagagcaggagaagttcctgacctgtgacgagtaccggggcaagcttcaggtgttcctgaggaccacgctgcgtcagtccgccacctcggccaccagctccaacgcgctctgggaggtggaggtggtccaccatgacccctgccgcggcggagcgggacactggaatggtctgtacaggttcaagcacctggccacgggcaactatctggcggctgaggaaaaccccagctacaagggtgatgtctcagatcctaaggcagcaggaccaggggcccagagccgcacaggccgcagaaacgccggcgagaagatcaagtaccgtctggtggctgtgccacatggcaacgacatcgcgtctctcttcgagctggaccccaccacgctgcagaaaaccgactccttcgtgccccggaactcctacgtgcggctgcggcacctctgtaccaacacctggatccagagcacgaacgcgcccatcgacgtggaggaggaacggcctatccggctcatgttgggcacgtgccccaccaaggaggacaaggaggcctttgccattgtgtcggttcccgtgtctgagatccgcgacctggactttgccaacgatgcaagctccatgctggccagcgctgtggagaaactcaacgagggcttcatcagccagaacgaccgcaggtttgtcatccagctgttggaggacttggtgttttttgtcagcgatgttcccaacaatgggcagaatgtcctggacatcatggtcaccaaacccaaccgagagcggcagaagctgatgcgggacgagaacatacttaagcagatctttggcatcctgaaggctccattccgtgacaaggggggtgaagggccattggtgcgtctggaggaactgtctgaccagaagaacgccccctaccagtacatgttccgcctgtgttaccgggtgctccggcactcccaggaggactaccgtaagaaccaggagcacatagccaagcagttcgggatgatgcagtcacagattggctatgacatcctggccgaagacaccatcacggccctgctccacaacaaccgaaaggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgcgactctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 3 - DNA, 31 bp, Artificial
  PCR oligo
ggagatctcgagctatgtctgacaaaatgtc

SEQ ID NO: 4 - DNA, 29 bp, Artificial
  PCR oligo
cgcggatcctttcggttgttgtggagcag

SEQ ID NO: 5 - DNA, 32 bp, Artificial
  PCR oligo
ggagatctcgagctatgaatgaaatgtccagc

SEQ ID NO: 6 - protein, 1082 aa, Artificial
  Synthetic Construct
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYKSGLRSRAMSDKMSSFLHIGDICSLYAEGSTNGFISTLGLVDDRCVVQPEAGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDQDASRSRLRNAQEKMVYSLVSVPEGNDISSIFELDPTTLRGGDSLVPRNSYVRLRHLCTNTWVHSTNIPIDKEEEKPVMLKIGTSPLKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKDPRVPVATMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 7 - protein, 1097 aa, Artificial
  Synthetic Construct
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYKSGLRSRAMNEMSSFLHIGDIVSLYAEGSVNGFISTLGLVDDRCVVEPAAGDLDNPPKKFRDCLFKVCPMNRYSAQKQYWKAKQTKQDKEKIADVVLLQKLQHAAQMEQKQNDTENKKVHGDVVKYGSVIQLLHMKSNKYLTVNKRLPALLEKNAMRVTLDATGNEGSWLFIQPFWKLRSNGDNVVVGDKVILNPVNAGQPLHASNYELSDNVGCKEVNSVNCNTSWKINLFMQFRDHLEEVLKGGDVVRLFHAEQEKFLTCDEYRGKLQVFLRTTLRQSATSATSSNALWEVEVVHHDPCRGGAGHWNGLYRFKHLATGNYLAAEENPSYKGDVSDPKAAGPGAQSRTGRRNAGEKIKYRLVAVPHGNDIASLFELDPTTLQKTDSFVPRNSYVRLRHLCTNTWIQSTNAPIDVEEERPIRLMLGTCPTKEDKEAFAIVSVPVSEIRDLDFANDASSMLASAVEKLNEGFISQNDRRFVIQLLEDLVFFVSDVPNNGQNVLDIMVTKPNRERQKLMRDENILKQIFGILKAPFRDKGGEGPLVRLEELSDQKNAPYQYMFRLCYRVLRHSQEDYRKNQEHIAKQFGMMQSQIGYDILAEDTITALLHNNRKDPRVPVATMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK